(19) United States
(12) Patent Application Publication
(10) Pub. No.: US 2026/0306025 A1
(43) Pub. Date: Oct. 8, 2026

(54) Brain Computer Interface (BCI) Device with a Flexible Connector

(71) Applicant: Echo Neurotechnologies Corporation, San Francisco, CA (US)

(72) Inventors: Kedar Gautam Shah, San Francisco, CA (US); Jason Ern-Chi Chung, San Francisco, CA (US)

(21) Appl. No.: 19/639,004

(22) Filed: Apr. 3, 2026

Related U.S. Application Data
(60) Provisional application No. 63/783,906, filed on Apr. 4, 2025.
(60) Provisional application No. 63/783,910, filed on Apr. 4, 2025.
(60) Provisional application No. 63/784,899, filed on Apr. 7, 2025.

(51) Int. Cl. A61B 5/00; G06F 3/01

(52) U.S. Cl. CPC A61B 5/686; G06F 3/015

(57) ABSTRACT

A cranial implant device includes a hermetically sealed package configured to be inserted into an opening in a skull of a patient. The device includes a set of electronic components enclosed within the hermetically sealed package. The device further includes a flexible electrode array configured to be positioned on a brain surface of the patient. The flexible electrode array includes a flexible substrate with electrode leads, a first portion positioned under and electrically coupled to a bottom surface of the hermetically sealed package, a second portion having a plurality of electrodes mounted on a surface of the flexible substrate, and a bent portion configured to bend the first portion with respect to the second portion, such that a subset of electrodes of the flexible electrode array is positioned under a back cover of the cranial implant device.

### RELATED APPLICATIONS

This application claims priority to (i) U.S. Provisional Application No. 63/783,906, filed Apr. 4, 2025, (ii) U.S. Provisional Application No. 63/783,910, filed Apr. 4, 2025, and (iii) U.S. Provisional Application No. 63/784,899, filed Apr. 7, 2025, each of which is incorporated by reference herein in its entirety.

This application is related to U.S. patent application Ser. No. ______ (Attorney Docket No. 141081-5004-US), filed Apr. 3, 2026, titled “Electrode Array Design for a Brain Computer Interface,” which is incorporated by reference herein in its entirety.

### TECHNICAL FIELD

The disclosed embodiments relate generally to brain computer interface (BCI), systems, and more specifically to BCI systems, devices, and methods for improved contact with the brain surface.

### BACKGROUND

Neurological conditions can result in the loss of communication and autonomy. Brain computer interface (BCI) technologies can assist individuals with neurological impairments, by recording and processing brain signals and translating the signals into outputs.

### SUMMARY

A brain computer interface (BCI) system can detect neural signals from a brain and decode the signals to determine an intended action such as an intent to speak or an intent to control a cursor, which is then to an output device such as a computer device, an audio or video output device, or a robotic arm.

In some scenarios, the BCI system can include an array of electrodes that is positioned on the surface of the sensorimotor cortex of the brain of a human subject, epidurally or subdurally, to obtain electrical activity from the surface of the brain. An electrode array that is inserted into the space between the skull and the cortex can move within the space, which may degrade the quality of the measured signals. Accordingly, there is a need for electrode array designs that resist movement and ensure good contact between the electrodes and the surface of the brain. In some scenarios, there is also a need to access the appropriate regions of the brain to obtain respective brain signals while minimizing the overall footprint of the implant device.

Some embodiments provide technical solutions to the aforementioned technical problems through the design and development of a flexible electrode array that facilitates accurate and reliable measurement of neural signals on the brain surface. Unlike some existing invasive approaches that involve electrodes penetrating into the brain, the present disclosure is directed to flexible, non-invasive electrode arrays that are positioned on a brain surface of a human subject (e.g., subdurally) and do not penetrate into the brain. Some embodiments disclose electrode arrays that include electrodes mounted on a flexible substrate and include an additional backing layer. As disclosed, in some embodiments, the backing layer provides a gap-filling structure by filling the subdural space that is normally filled with cerebrospinal fluid. The backing layer facilitates a slight downward pressure on the electrode array and improves electrode contact with the brain. For example, the backing layer functions can keep the electrodes in contact with the brain during brain pulsation (e.g., due to respiratory/cardiac cycles) or other brain motion. In some embodiments, a portion of the flexible electrode is curved with a radius of curvature that approximates a curvature of the brain. The radius of curvature is selected so that the flexible electrode creates a downward force that keeps the electrodes in contact with the brain during brain pulsation. In some embodiments, the combination of the curved portion of the flexible electrode and the backing layer facilitates a slight downward pressure on the electrode array and improves electrode contact with the brain. By improving contact with the brain, higher quality neural signals can be collected. In some embodiments, the backing layer increases stiffness of the electrode array to help guide the array under the dura during implantation of the cranial implant device. In some embodiments, the backing layer may be adjustable, inflatable, or compressible to match patient anatomy or local brain geometry. Some embodiments describe electrodes that are flushed with a substrate or stand ‘proud’ of a lead surface, which allow the electrodes to be closer to the brain for making more intimate contact with the brain surface, thereby improving the quality of the acquired signals.

Some embodiments disclose an implant device that is configured to maximize the coverage of a sensorimotor cortex region of a patient while minimizing the total footprint of the implant device, by routing electrodes of the electrode array underneath a hermetically sealed package of electronic components. Positioning the electrodes (or a subset thereof) underneath the hermetically sealed package allows neural signals that are directly under the hermetically sealed package to be collected while reducing the area of a skull required to be removed during craniectomy.

In accordance with some embodiments, a cranial implant device is configured to be inserted into an opening in a skull of a patient. The cranial implant device includes an electrode array configured to be positioned on a brain surface of a patient. The electrode array includes a flexible substrate with electrode leads. The electrode array further includes a plurality of electrodes mounted on and protruding from the flexible substrate such that at least some of the plurality of electrodes are configured to be proximate to the brain surface of the patient when positioned in the patient.

In accordance with some embodiments, a cranial implant device is configured to be inserted into an opening in a skull of a patient. The cranial implant device includes an electrode array configured to be positioned on a brain surface of a patient. The electrode array includes a plurality of electrodes positioned at a first side of the electrode array. The electrode array includes a backing layer adapted to reduce a space between a skull and the brain of the patient thereby improving contact between the electrodes and the brain surface. In some embodiments, the backing layer functions as a gap-filling structure by filling the subdural space that is normally filled with cerebrospinal fluid. In some embodiments, the backing layer facilitates a slight downward pressure on the electrode array and improves electrode contact with the brain. In some embodiments, the backing layer is adapted to reduce (e.g., limit or prevent) relative motion of the electrode array on the brain surface of the patient. In some embodiments, the backing layer is coupled to a second side of the electrode array, opposite to the first side. In some embodiments, one or more intermediate layers, different from the backing layer, are coupled to the second side of the electrode array, and the backing layer is coupled to at least one of the one or more intermediate layers.

In accordance with some embodiments, a cranial implant device includes a hermetically sealed package configured to be inserted into an opening in a skull of a patient. The cranial implant device further includes a set of electronic components enclosed within the hermetically sealed package. The cranial implant device further includes a flexible electrode array configured to be positioned on a brain surface of the patient. The flexible electrode array includes (i) a flexible substrate with electrode leads; (ii) a first portion positioned under and electrically coupled to a bottom surface of the hermetically sealed package; (iii) a second portion having a plurality of electrodes mounted on a surface of the flexible substrate; and (iv) a bent portion configured to bend the first portion with respect to the second portion such that a subset of electrodes of the flexible electrode array is positioned under a back cover of the cranial implant device.

In accordance with some embodiments, a method of inserting a cranial implant device into an opening in a skull of a patient is disclosed. The cranial implant device includes an electrode array having an opening at an interior portion of the electrode array. The method includes making an incision into a dura mater layer of the patient. The method includes inserting a first portion, less than all, of the electrode array under the dura mater layer of the patient via the incision, such that the opening at the interior portion of the electrode array is located at the dural incision. The method includes fastening the dura mater layer at the opening. In some embodiments, fastening the dura mater includes applying one or more of suturing, clipping or other surgical methods of reapproximating the dura.

In accordance with some embodiments, a method of inserting a cranial implant device into an opening in a skull of a patient is disclosed. The cranial implant device includes an electrode array having a bent portion. The method includes making a slit (e.g., an incision) into a dura mater layer of the patient. The method further includes unbending the bent portion of the electrode array to form an unbent portion. The method further includes inserting at least a part of the electrode array under the dura mater layer of the patient via the slit. The method further includes fastening (e.g., by suturing) the dura mater layer. The method further includes re-bending the unbent portion of the electrode array. The method further includes anchoring the cranial implant device to the skull of the patient via a plurality of screws.

In accordance with some embodiments, a computing device includes a display, one or more processors, and memory coupled to the one or more processors. The memory stores one or more programs configured for execution by the one or more processors. The one or more programs include instructions for performing any of the methods disclosed herein.

In accordance with some embodiments, a computer system includes one or more processors, and memory coupled to the one or more processors. The memory stores one or more programs configured for execution by the one or more processors. The one or more programs include instructions for performing any of the methods disclosed herein.

In accordance with some embodiments, a non-transitory computer readable storage medium stores one or more programs configured for execution by a computing device having a display, one or more processors, and memory. The one or more programs include instructions for performing any of the methods disclosed herein.

In accordance with some embodiments, a non-transitory computer readable storage medium stores one or more programs configured for execution by a computer system having one or more processors, and memory. The one or more programs include instructions for performing any of the methods disclosed herein.

Thus systems, devices, and methods are disclosed that improve the quality of acquired neural signals and maximize the coverage of a sensorimotor cortex region of a patient while minimizing the total footprint of the implant device.

Note that the various embodiments described above can be combined with any other embodiments described herein. The features and advantages described in the specification are not all inclusive and, in particular, many additional features and advantages will be apparent to one of ordinary skill in the art in view of the drawings, specification, and claims. Moreover, it should be noted that the language used in the specification has been principally selected for readability and instructional purposes and may not have been selected to delineate or circumscribe the inventive subject matter.

### BRIEF DESCRIPTION OF THE DRAWINGS

For a better understanding of the aforementioned systems, methods, and graphical user interfaces, reference should be made to the Detailed Description of Embodiments below, in conjunction with the following drawings in which like reference numerals refer to corresponding parts throughout the figures. The accompanying drawings, which are included to provide a further understanding of the embodiments, are incorporated herein, constitute a part of the specification, illustrate the described embodiments, and, together with the description, serve to explain the underlying principles.

FIG. 1A illustrates an example operating environment, in accordance with some embodiments.

FIG. 1B illustrates example operating modes of a computing device, in accordance with some embodiments.

FIG. 1C is a graph showing an actual time-series dataset of neural activity of a patient, in accordance with some embodiments.

FIG. 1D is a graph showing neural features extracted from the neural activity dataset of FIG. 1C, in accordance with some embodiments.

FIGS. 2A, 2B, and 2C illustrate an example implant device, in accordance with some embodiments.

FIG. 2D is a block diagram showing an example data path, control path, and power path of an implant device, in accordance with some embodiments.

FIG. 3A illustrates an example wearable device, in accordance with some embodiments.

FIG. 3B is a block diagram showing an example data path, control path, and power path of a wearable device, in accordance with some embodiments.

FIG. 4 is a block diagram illustrating an example computing device in accordance with some embodiments.

FIG. 5 is a block diagram illustrating an example server system in accordance with some embodiments.

FIGS. 6A to 6C illustrate example electrode arrays of an implant device, in accordance with some embodiments.

FIGS. 6D-1 to 6D-4 are side views of an electrode array that includes a flexible substrate with fins, in accordance with some embodiments.

FIGS. 7A to 7C illustrate example electrode configurations implemented in an electrode array, in accordance with some embodiments.

FIGS. 8A to 8D illustrate an electrode array with a backing layer and placement of the electrode array relative to a brain surface of a patient, in accordance with some embodiments.

FIG. 9 illustrates an example implant device including a hermetically sealed package and a bent electrode array featuring a flexible connector, in accordance with some embodiments.

FIGS. 10A-1, 10A-2, 10A-3, and 10B illustrate an example process of forming a bent portion featuring a 180-degree bend, in accordance with some embodiments.

FIG. 10C illustrates a view of the implant device with a flexible electrode array, in accordance with some embodiments.

FIG. 11 illustrates an example process of forming a bent portion featuring a 90-degree bend, in accordance with some embodiments.

FIG. 12A illustrates an example process of inserting an implant device into an opening in a skull of a patient, in accordance with some embodiments.

FIG. 12B is a cross-sectional view depicting the incision on the dura and the insertion of the flexible electrode array, in accordance with some embodiments.

FIG. 12C illustrates exemplary positions of the sutures relative to the flexible electrode array according to some embodiments.

FIG. 12D illustrates contact of the implant device with bone and bodily fluids of a human subject in accordance with some embodiments.

FIG. 13 illustrates a flow diagram illustrating a method of inserting a cranial implant device into an opening in a skull of a patient, in accordance with some embodiments.

FIG. 14 illustrates a flow diagram illustrating a method of inserting a cranial implant device into an opening in a skull of a patient, in accordance with some embodiments.

Like reference numerals refer to corresponding parts throughout the several views of the drawings.

### DETAILED DESCRIPTION OF EMBODIMENTS

Reference will now be made in detail to specific embodiments, examples of which are illustrated in the accompanying drawings. In the following detailed description, numerous non-limiting specific details are set forth in order to assist in understanding the subject matter presented herein. But it will be apparent to one of ordinary skill in the art that various alternatives may be used without departing from the scope of the claims and the subject matter may be practiced without these specific details. For example, it will be apparent to one of ordinary skill in the art that the subject matter presented herein can be implemented on many types of electronic devices with digital video capabilities.

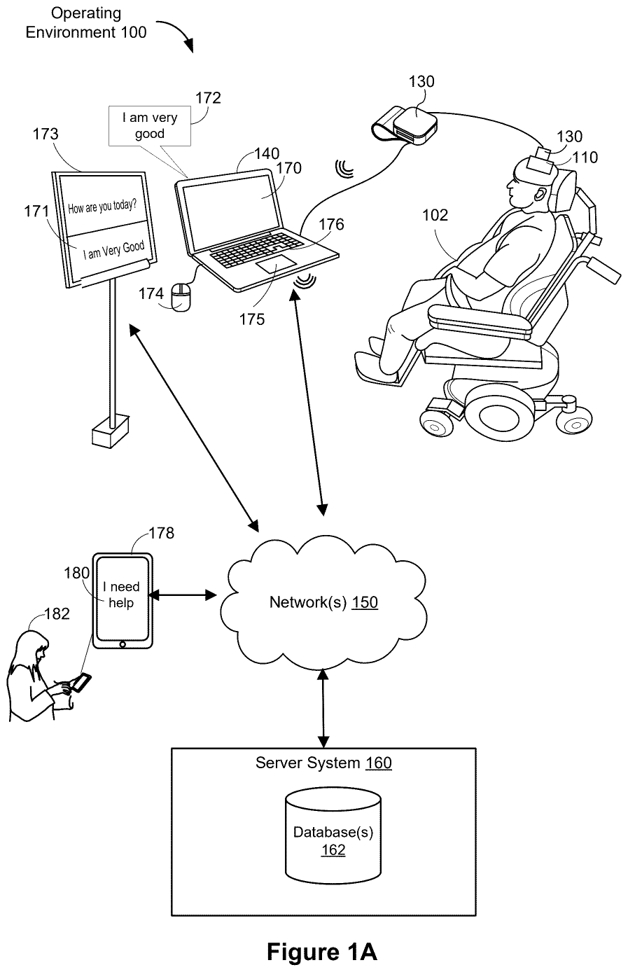

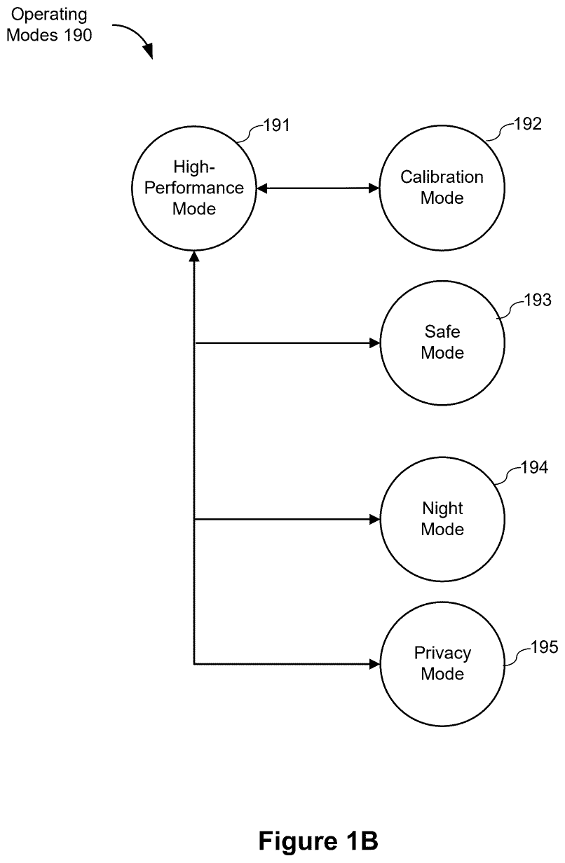

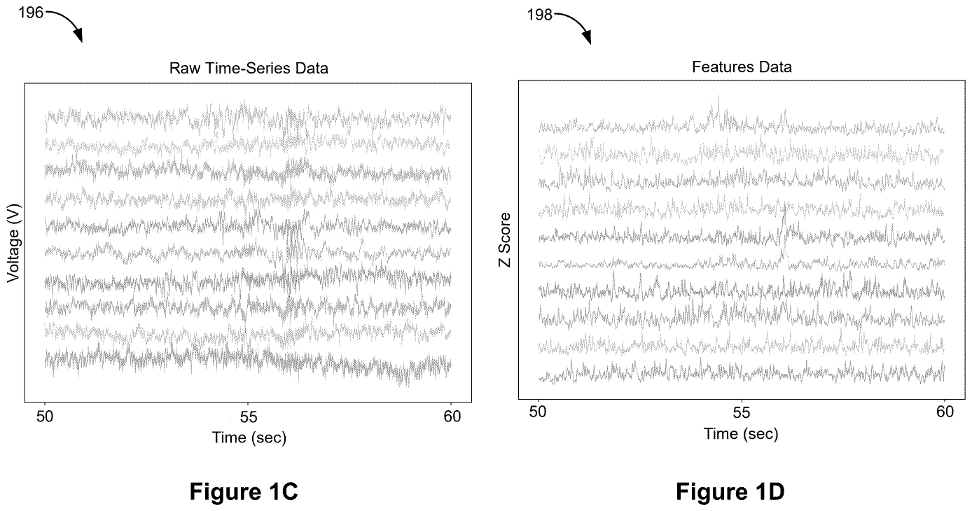

FIG. 1A illustrates an example operating environment 100 of a brain computer interface (BCI) system, in accordance with some embodiments.

As used herein, a BCI system is a system that acquires brain signals from a user and determines, from the signals, into one or more intended actions (e.g., functional intents) of the user. In some embodiments, the BCI system generates and executes commands for controlling one or more external devices without requiring traditional muscle movements of the user. Because no user movement or vocalization is required to control the BCI system, the BCI system can assist users with disabilities, thereby enhancing the user's capabilities and improving their quality of life.

The operating environment 100 depicts a human subject 102. As used herein, human subject 102 is also referred to as a “patient” or a “user.” In some instances, the human subject 102 is unable to speak or unable to move their limbs. In some embodiments, the BCI system includes an implant device 110 (also referred to herein as a cranial implant device), which is implanted into the skull of the human subject 102. The implant device 110 includes an array of high-density electrode sensors for recording neural signals (e.g., electrical signals) corresponding to motor cortex activity directly from the brain surface of the human subject 102. In some embodiments, the array is a brain-penetrating electrode array or an electrocorticography (ECoG) electrode array. In some embodiments, the electrodes are depth electrodes or surface electrodes. In some embodiments, the recorded neural signals represent specific activities of the brain, including attempted speech, attempted movement, hearing, and/or vision. In some embodiments, the array of electrode sensors are placed on the cortical surface of the brain, either epidurally or subdurally, to record signals directly from the brain. In some embodiments, the array of electrode sensors can penetrate under the scalp.

In some embodiments, the BCI system includes a wearable device 130. As described in further detail in FIG. 3A, in some embodiments the wearable device 130 includes a patch component that is magnetically and communicatively coupled to the implant device 110. The patch component is configured to transmit power wirelessly into the implant device 110 and receive neural signal data from the implant device 110. In some embodiments, the wearable device 130 includes a relay component that includes electronics for supporting the functionalities of the patch component.

In some embodiments, the BCI system includes a computing device 140 that is communicatively connected with the wearable device 130. The computing device 140 can include a display 170, a keyboard 176 and a pointing device such as a mouse 174 or a touchpad 175. In accordance with some embodiments, the computing device 140 functions as the main interface for the human subject 102. For example, the human subject 102 can access one or more applications executing on the computing device 140 for their daily tasks and activities such as checking email and browsing the web. In some embodiments, the one or more applications executing on the computing device 140 includes a BCI application (e.g., application 440) that is configured to decode the neural signals and interpret the decoded signals to determine one or more intended actions (e.g., functional intents) of the human subject 102. In some embodiments, the BCI application applies one or more machine learning models that are stored locally on the computing device 140 to perform the decoding and interpreting. In some embodiments, the BCI application is configured to execute on the computing device 140 even while the computing device 140 is offline (although an internet connection will be required to receive new models from the server system 160).

In some embodiments, the BCI system includes a server system 160 that is communicatively coupled to the computing device 140 through one or more communication networks 150. The server system 160 includes an operational pipeline that is configured to automatically generate different machine learning models based on respective input data, train the models to optimize their performance on specific tasks, and deploy the models locally on respective computing devices 140. In some embodiments, the server system 160 includes one or more databases 162 for storing data such as raw data obtained from implanted devices corresponding to respective patients, feature data, analytics data, application information, and firmware versions.

With continued reference to FIG. 1A, in some embodiments, the BCI system is configured to control one or more output devices to carry out one or more intended actions of the human subject 102 according to the decoded signals. As one example, in some embodiments, the BCI system (e.g., computing device 140 or server system 160) is configured to control a keyboard (e.g., physical keyboard 176 or a virtual keyboard) to generate language and/or numeral outputs according to the decoded signals (e.g., by typing letters, numbers, words and/or sentences using the keyboard), without requiring any voice command and/or limb movement by the human subject 102. As another example, in some embodiments, the BCI system is configured to control a cursor (e.g., by controlling a pointing device such as mouse 174, touchpad 175, or the cursor) according to the decoded signals, without requiring any voice command and/or limb movement by the human subject 102. As another example, in some embodiments, the BCI system is configured to control a display device (e.g., display 170 or external display device 173) to display a visual output (e.g., text entry 171) corresponding to a response from the human subject 102, without requiring any voice command and/or limb movement by the human subject 102. As another example, in some embodiments, the BCI system is configured to translate a visual/text output into a verbal expression and generate an audio output 172 articulating the verbal expression using an audio output component of the computing device 140, without requiring any voice command and/or limb movement by the human subject 102. As another example, in some embodiments, the BCI system is configured to control one or more interfaces of the computing device 140 to navigate through menus and/or switch between tabs of one or more applications executing on the computing device 140, without requiring any voice command and/or limb movement by the human subject 102. As yet another example, in some embodiments, the BCI system is configured to generate an alert according to the decoded signals and transmit the alert to an electronic device 178 that is associated with a caregiver 182 of the human subject 102, for display (e.g., as alert message 180) on a display screen of the electronic device 178.

FIG. 1B illustrates example operating modes 190 of a computing device 140, in accordance with some embodiments.

In accordance with some embodiments, the computing device 140 can switch between various operating modes. A respective operating mode may have its associated (e.g., distinct) user interface. A human subject 102 may interact with the computing device 140 in specific ways, depending on the operating mode.

Referring to FIG. 1B, in some embodiments, the operating modes include a high performance mode 191 that allows a human subject 102 to fully interact with the computing device 140. For example, in the high performance mode 191, the human subject 102 has access to all functionalities of applications that are executing on the computing device. In some embodiments, the high performance mode 191 is also referred as the “normal” mode. In some embodiments, high performance mode 191 is the default operating mode of the computing device.

In some embodiments, the operating modes include a calibration mode 192, which is a sandbox mode used during training tasks.

In some embodiments, the operating modes include a safe mode 193. In some embodiments, the safe mode 193 is a fallback mode for when the high performance mode 191 is not available.

In some embodiments, the operating modes include a night mode 194. When operating in the night mode, the display screen 170 of the computing device 140 is not in use and the computing device 140 has limited functionality due to the screen not being in use.

In some embodiments, the operating modes include a privacy mode 195, where brain signals are not recorded and not decoded.

FIG. 1C is a graph 196 showing an actual raw time-series dataset of neural activity of a patient collected by an electrode array, in accordance with some embodiments. The dataset is collected by placing electrodes on the surface of the patient's brain. Each of the line plots in the graph 196 corresponds to electrical field signals collected by a respective channel (e.g., an electrode) of the array. The line plots on the graph 196 have been offset for clarity.

FIG. 1D is a graph 198 showing a neural-feature time-series dataset extracted from the neural activity data of FIG. 1C, in accordance with some embodiments. In some embodiments, the neural-feature time series dataset is generated using a feature processing pipeline that is executed by the computing device 140 (e.g., application 440) or the server system 160 (e.g., operational pipeline 524). The neural features correspond to representations of brain signals that contain information related to underlying neural processing, including neural representations associated with attempted speech and/or motor movements. The values on the y-axis of the graph 198 are z-score values, which are normalized values that indicate how many standard deviations a data point is from the mean of the dataset (computed individually for each feature time series).

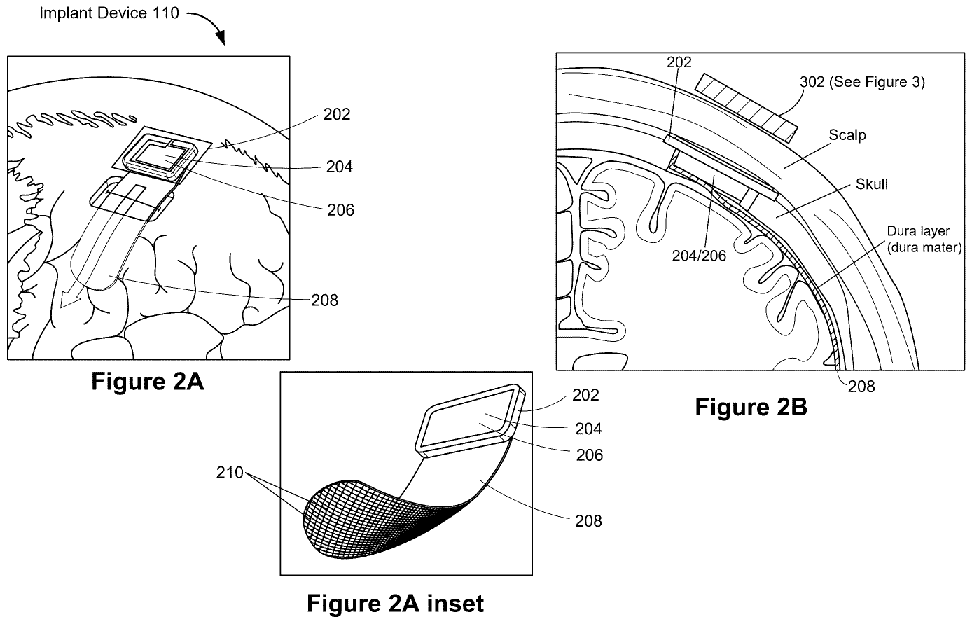

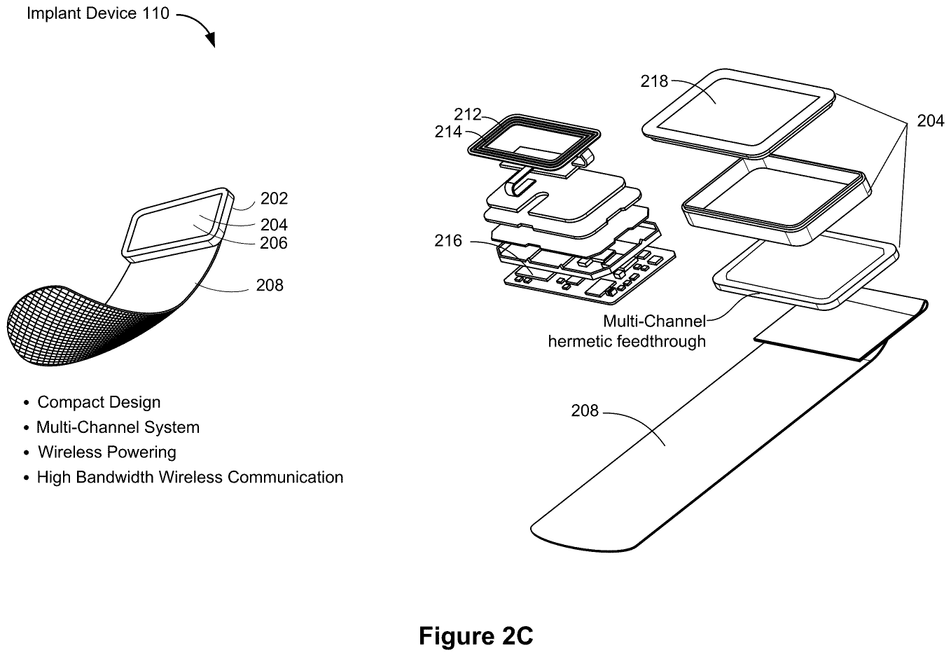

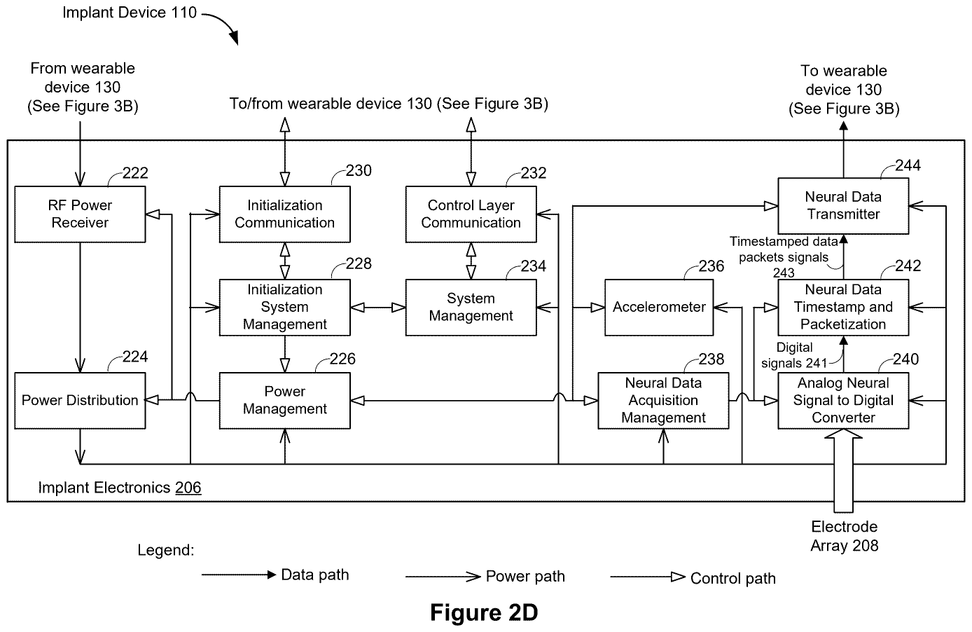

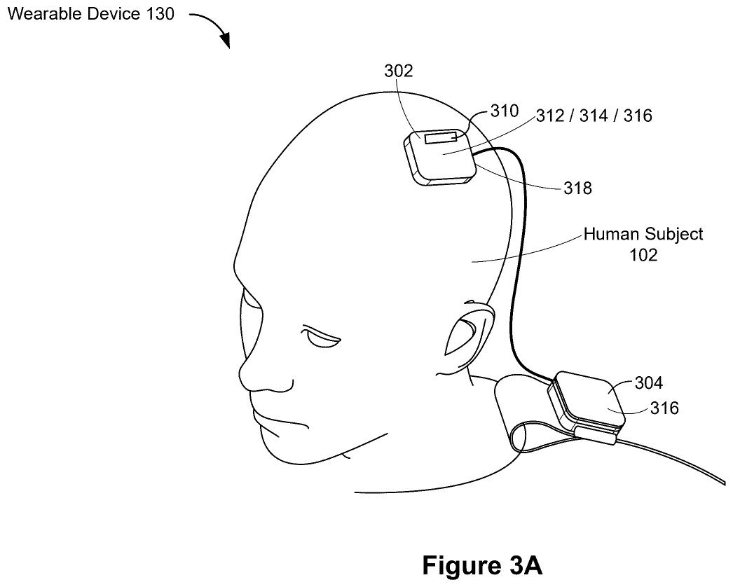

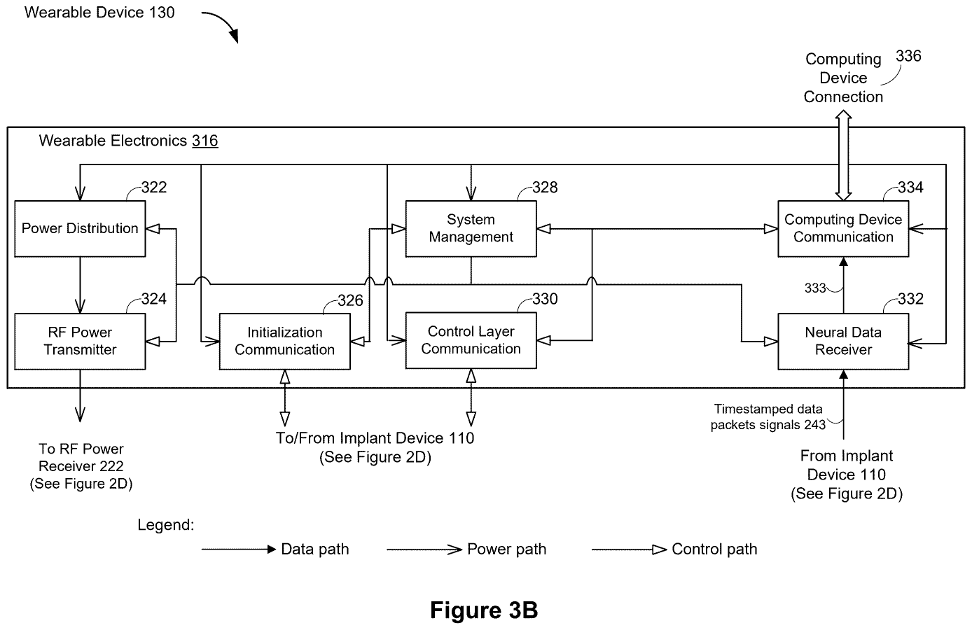

FIGS. 2A, 2B, and 2C illustrate various views of the implant device 110, in accordance with some embodiments.

In some embodiments, the implant device 110 includes a frame 202, a hermetic package 204 (e.g., hermetically-sealed package), implant electronics 206 (e.g., one or more electronic components) enclosed in the hermetic package 204, and an electrode array 208. As used herein, the electrode array 208 is also referred to as “an array of electrodes” or “an array of electrode sensors.” FIG. 2A inset shows that the electrode array 208 comprises a thin, flexible array (e.g., with a thickness of around 50 μm to 200 μm) that includes multiple electrode sensors 210. In some embodiments, the electrode array 208 comprises an electrocorticography (ECoG) array that is surgically implanted on the brain's surface.

In some embodiments, the electrode array 208 includes at least 100 electrodes, at least 250 electrodes, at least 500 electrodes, at least 750 electrodes, at least 1000 electrodes, at least 1500 electrodes, at least 2000 electrodes, at least 2500 electrodes, at least 3000 electrodes, at least 4000 electrodes, at least 5000 electrodes, at least 7500 electrodes, or at least 10,000 electrodes. In some embodiments, the electrode array 208 is designed to be placed over a substantial portion of or the entire sensorimotor cortex region, to collect electrophysiologic data with information content of intended speech and movement of a human subject. In some embodiments, the electrode array is designed to be placed over one or more subregions or regions of the cortex, such as the precentral gyrus, postcentral gyrus, posterior superior frontal gyrus, posterior middle frontal gyrus, posterior inferior frontal gyrus region, superior temporal gyrus, or a combination of any of the foregoing. Typically, each electrode 210 of the electrode array 208 corresponds to a channel and is configured to pick up electrical activity from the cortex. A denser electrode array with higher channel count can capture more signals with higher fidelity, which can be used to decode intended actions (e.g., intended speech actions and non-speech actions) of the human subject.

FIG. 2B illustrates a coronal cross-sectional view of a brain of a human subject. In some embodiments, the frame 202 is configured to be anchored to a skull of a human subject 102 via a surgical procedure called craniectomy, in which a scalp incision is made and a full-thickness section of bone is removed from the skull. In the example of FIG. 2B, the frame 202, the hermetic package 204, and the implant electronics 206 are configured to be positioned above the dura layer (e.g., closer to the scalp), whereas the electrode array 208 is inserted into the sub-dural layer (e.g., underneath the dura mater) of the brain of the human subject 102. During device operation, the electrode array 208 is configured to acquire (e.g., record) motor cortex neural signals (e.g., electrical signals) from the brain of the human subject.

In some embodiments, the implant device 110 includes a battery. In some embodiments, the implant device 110 does not include a battery. In some embodiments, the implant device 110 is configured to receive power signals from a patch component 302 of the wearable device 130. In some embodiments, the implant electronics 206 include a wireless powering coil 212 that is configured to receive electrical power wirelessly from the patch component 302. In some embodiments, the hermetic package 204 includes a cover 218 that is composed of a ceramic material or other dielectric material to facilitate the receiving of power signals via inductive coupling or capacitive coupling. In some embodiments, the wireless powering coil 212 is configured to receive constant electrical power from the patch component 302.

FIG. 2B illustrates coupling between the implant device 110 and the patch component 302. In some embodiments, the frame 202 includes one or more magnets that are configured to magnetically couple to one or more complementary magnets positioned at the patch component 302.

In some embodiments, the implant electronics 206 include a wireless communication antenna 214 that is configured to establish a direct wireless link between the implant device 110 and the wearable device 130. In some embodiments, the wireless communication antenna 214 is configured to implement high bandwidth wireless communication to transmit the multiple channels (e.g., hundreds or thousands of channels) of brain signals (e.g., electrical signals) that are collected by the electrode array 208 to the wearable device 130. In some embodiments, the implant device 110 is configured to amplify, digitize, and transmit the signals to the patch component 302 as data packets without interpreting and/or further processing the signals.

In some embodiments, the implant electronics 206 include processing circuitry, communication circuitry, and power circuitry. In some embodiments, the various circuitries are implemented as a multi-channel application-specific integrated circuit (ASIC) chip 216 that enables custom programming of specific tasks. In some embodiments, the ASIC chip 216 is implemented as a system-on-chip (SOC). In some embodiments, the ASIC chip 216 and includes one or more microprocessors and one or more memory blocks such as ROM, RAM, EEPROM, flash memory, or other building blocks.

FIG. 2D is a block diagram showing an example data path (represented by closed triangle arrows), power path (represented by two-edged arrows), and control path (represented by open triangle arrows) of an implant device 110, in accordance with some embodiments.

The data path of the implant device 110 includes receiving neural signals from the electrode array 208. In some embodiments, the implant device 110 includes an analog neural signal to digital converter module 240 (e.g., an analog to digital converter) for converting the neural signals (e.g., electrical signals) from analog form to digital form (e.g., digital signals 241). The implant device 110 includes a neural data timestamp and packetization module 242 for embedding date/time information to the digital signals and breaking down the digital signals into data packets (e.g., data segments) of smaller data sizes (e.g., timestamped data packets 243). The implant device 110 includes a neural data transmitter module 244 for transmitting the timestamped data packets 243 to the wearable device 130.

In accordance with some embodiments, the implant device 110 does not include a battery (i.e., the implant device 110 is not a battery-operated device). In some embodiments, the implant device 110 receives its power supply from the wearable device 130. In some embodiments, the implant device 110 includes an RF power receiver module 222 for receiving power (e.g., DC power) from the wearable device 130 (e.g., from RF power transmitter module 324 of the wearable device 130) and sending the received power to a power distribution module 224. The power distribution module 224 is configured to distribute the power to the various modules of the implant device 110, including the analog neural signal to digital converter module 240, the neural data timestamp and packetization module 242, the neural data transmitter module 244, a power management module 226, an initialization system management module 228, an initialization communication module 230, a control layer communication module 232, a system management module 234, an accelerometer module 236, and a neural data acquisition module 238.

In some embodiments, the power management module 226 is configured to actively control and monitor the flow of electrical power within the implant device 110. For example, the power management module 226 can regulate voltage, manage current draw, and protect modules such as the neural data acquisition module 238, the analog neural signal to digital converter module 240, the neural data timestamp and packetization module 242, the neural data transmitter module 244, and the accelerometer module 236 against overloads, thus ensuring efficient and safe power distribution to modules or components of the implant device 110.

In some embodiments, the initialization system management module 228 is configured to handle the initialization process when the implant device 110 first starts up, by coordinating the loading and configuration of settings and modules, such as the initialization communication module 230, the system management module 234, and the power management module 226.

In some embodiments, the initialization communication module 230 is configured to set up and prepare the communication channels and protocols needed for the implant device 110 to interact with the wearable device 130 at the beginning of its operation.

In some embodiments, the control layer communication module 232 is configured to manage the flow of data between different modules of the implant device 110 and between the implant device 110 and the wearable device 130.

In some embodiments, the system management module 234 is configured to manage and monitor the performance, health, and operation of the implant device 110. For example, the system management module 234 can track system parameters such as temperature, voltage, and power consumption to ensure stable operation of the implant device 110. In some instances, the system management module 234 can identify system failures, log errors, and provide alerts for troubleshooting.

In some embodiments, the accelerometer module 236 (e.g., hardware accelerator) is specifically designed to significantly speed up the execution of specific tasks by offloading them from the main processor of the ASIC chip 216.

In some embodiments, the neural data acquisition module 238 is configured to record electrical activity from the brain (e.g., neurons) using electrodes 210 of the electrode array 208. In some embodiments, the neural data acquisition module 238 is configured to acquire the electrical activity without performing additional processing on the recorded data. In some embodiments, the neural data acquisition module 238 is configured to boost weak neural signals. In some embodiments, the neural data acquisition module 238 is configured to filter out noise and unwanted frequencies to improve signal quality.

FIG. 3A illustrates a wearable device 130, in accordance with some embodiments. In some embodiments, the wearable device 130 includes a patch component 302, a relay component 304, and wearable electronics 316 (e.g., electronic components).

In some embodiments, the patch component 302 includes one or more magnets 310 that are configured to magnetically couple and align the patch component 302 to the implant device 110. In some embodiments, the patch component 302 includes one or more coils 312 and/or antenna 314 for transmitting power wirelessly into the implant device 110, transmitting command signals into the implant device 110, and/or receiving neural signal data transmitted from the implant device 110. In some embodiments, the patch component 302 includes wearable electronics 316 (e.g., electronic components) (e.g., to power the coils 312 and/or the antennas 314. In some embodiments, the patch component 302 includes a mechanical housing 318 for holding the magnets 310 the coils 312, the antenna 314, and/or the wearable electronics components 316.

In some embodiments, the relay component 304 includes additional electronics for supporting functionalities of the patch component 302, and for enabling data and power transfer between the patch component 302 and the computing device 140. In some embodiments, the relay component 304 is configured to anchor the wearable device 130 on or near the human subject 102, such as on clothing worn by the human subject, or on a wheelchair or mobility device of the human subject, or on a patient bed of the human subject.

FIG. 3B is a block diagram showing an example data path (represented by closed triangle arrows), power path (represented by two-edged arrows), and control path (represented by open triangle arrows) of a wearable device 130, in accordance with some embodiments.

Data path-wise, in some embodiments, the wearable device 130 includes a neural data receiver module 332 for receiving neural signals in the form of timestamped data packets 243 from the implant device 110. In some embodiments, the neural data receiver module 332 generates packetized combined data 333 by further packetizing the timestamped data packets 243 along with data from the wearable device 130 and control commands. The neural data receiver module 332 transmits the packetized combined data 333 to a computing device communication module 334 that is configured to transmit the packetized combined data 333 to the computing device 140 via a connection 336 of the computing device.

The wearable device 130 is connected to an electrical power source. For example, the wearable device 130 can be connected to an electrical power outlet or can include an energy storage device and/or a battery that is configured to supply power to the wearable device 130. In some embodiments, the wearable device 130 includes a RF transmitter module 324 that is configured to transmit electrical power to the implant device 110 (e.g., to the RF power distribution module 322). Ad depicted in FIG. 3B, the electrical power source of the wearable device 130 is configured to provide power to various modules of the wearable electronics 316, including an initialization communication module 326, a system management module 328, a control layer communication module 330, the neural data receiver module 332, and the computing device communication module 334.

In some embodiments, the initialization communication module 326 is configured to set up and prepare the communication channels and protocols needed for the wearable device 130 to interact with the implant device 110 at the beginning of its operation.

In some embodiments, the system management module 328 is configured to manage and monitor the performance, health, and operation of the wearable device 130. For example, the system management module 328 can track system parameters such as temperature, voltage, and power consumption to ensure stable operation of the wearable device 130. In some instances, the system management module 328 can identify system failures, log errors, and provide alerts for troubleshooting.

In some embodiments, the control layer communication module 330 is configured to manage the flow of data between different modules of the wearable device 130 and between the wearable device 130 and the implant device 110.

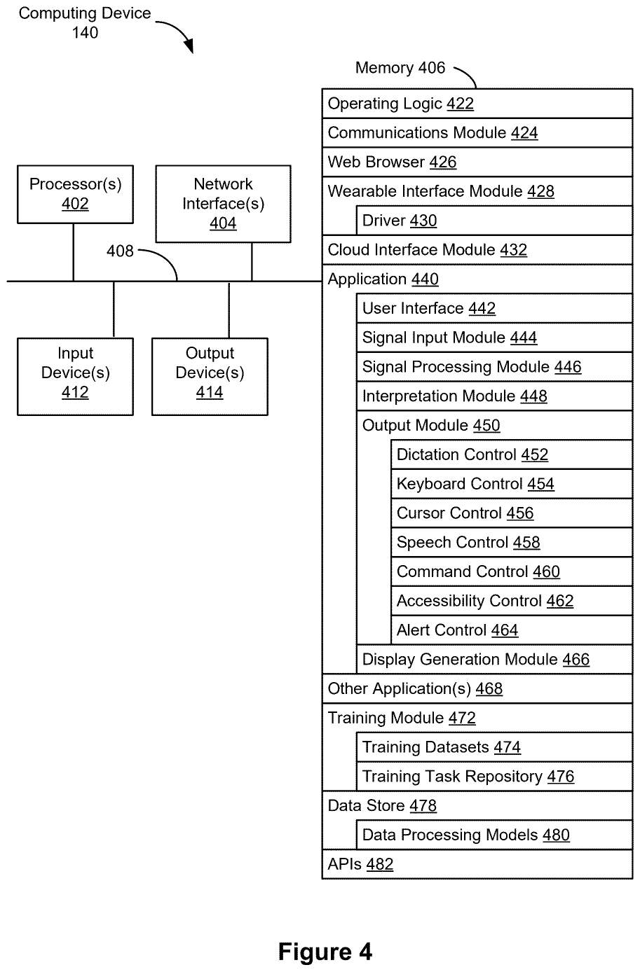

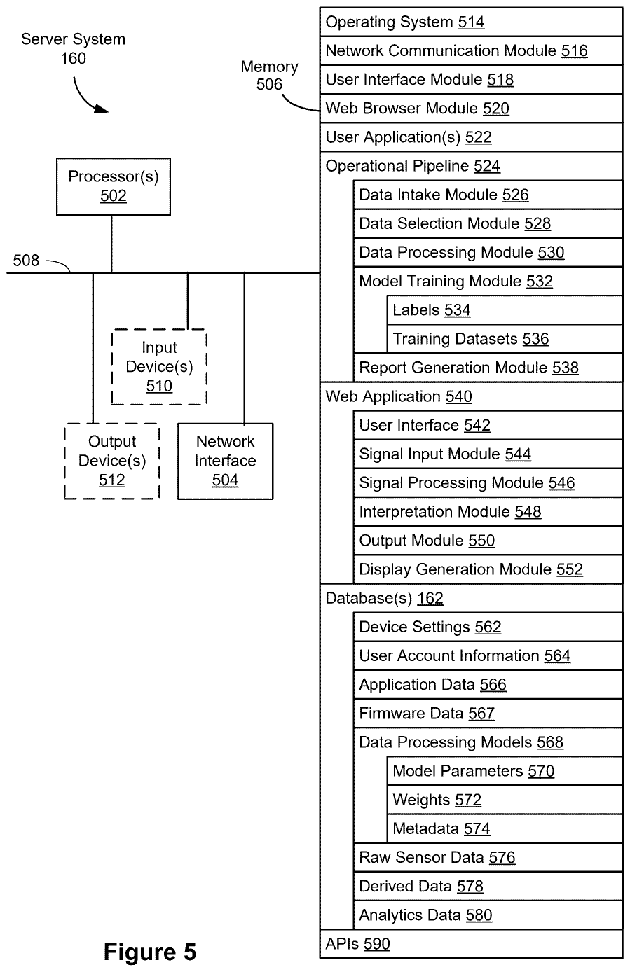

FIG. 4 is a block diagram illustrating a computing device 140, in accordance with some embodiments. Various examples of the computing device 140 include a desktop computer, a laptop computer, a tablet computer, a virtual reality (VR) device, an augmented reality (AR) device, or a spatial computing device that blends digital content with the physical world. The computing device 140 includes one or more processors 402 (e.g., processing units (CPUs) or cores), one or more network or communication interfaces 404, memory 406, and one or more communication buses 408 for interconnecting these components. In some embodiments, the communication buses 408 include circuitry (sometimes called a chipset) that interconnects and controls communications between system components. In some embodiments, the computing device 140 includes a user interface 410. The user interface 410 typically includes a display (e.g., display device) 412.

In some embodiments, the computing device 140 includes one or more input devices 412, which facilitate user input, such as a keyboard, a mouse, a voice-command input unit or microphone, a display 170 (e.g., which can be a touch-sensitive display), a touch-sensitive input pad, a gesture capturing camera, or other input buttons or controls. In some embodiments, the computing device 140 uses a microphone and voice recognition or a camera and gesture recognition to supplement or replace the keyboard. In some implementations, the computing device 140 includes one or more cameras, scanners, or photo sensor units for capturing images. In some embodiments, the computing device 140 includes one or more output devices 414, which enable presentation of user interfaces and display content, including one or more speakers and/or one or more visual displays.

The memory 406 includes high-speed random-access memory, such as DRAM, SRAM, DDR RAM, or other random-access solid-state memory devices. In some embodiments, the memory 20406 6 includes non-volatile memory, such as one or more magnetic disk storage devices, one or more optical disk storage devices, one or more flash memory devices, or one or more other non-volatile solid state storage devices. In some embodiments, the memory 406 includes one or more storage devices remotely located from the processors 402. The memory 406, or alternatively the non-volatile memory devices within the memory 406, includes a non-transitory computer-readable storage medium. In some embodiments, the memory 406, or the computer-readable storage medium of the memory 406, stores the following programs, modules, and data structures, or a subset or superset thereof:
an operating system 422, which includes procedures for handling various basic system services and for performing hardware dependent tasks;
a communications module 424, which connects the computing device 140 to other devices (e.g., various servers in the server system 160, implant device 110, wearable device 130, external display device 173, and mouse 174) via one or more network interfaces 404 (wired or wireless) and one or more networks 150, such as the Internet, other wide area networks, local area networks, metropolitan area networks, and so on;
a web browser 426 (or other application capable of displaying web pages), which enables a user to communicate over a network with remote computers or devices;
a wearable interface module 428, for interfacing with various modules and components of the wearable device 130. In some embodiments, the wearable interface module 428 includes:
   a driver 430, which provides two-way telemetry between the computing device and the wearable device 130 and data input for application 440;
a cloud interface module 432, for providing two-way communication between the computing device 140 and the server system 160;
an application 440. In some embodiments, the application 440 includes:
   a user interface 442 for providing visual information to users and for capturing user inputs;
   a signal input module 444 for receiving brain signals (e.g., electrical signals or data streams) of a human subject 102. As discussed with reference to FIGS. 1, 2, and 3, in some embodiments, the brain signals are collected by implant device 110 and transmitted to the wearable device 130. In some embodiments, the brain signals include attempted or intended speech signals (e.g., the human subject 102 tries to speak, thereby producing electrical signals corresponding to the intended movement of specific individual muscles or groups of muscles used for speech articulation, but their speech may not be vocalized due to underlying pathology preventing correct movement of the intended muscles or muscle groups). In some embodiments, the brain signals include attempted non-speech motor signals, such as attempted head, arm, hand, foot or leg movement signals (e.g., the human subject 102 tries to move their head or limbs, thereby producing electrical signals corresponding to the intended movement of specific individual muscles or groups of muscles used for the desired movement, but actual, physical movement may or may not occur due to underlying pathology preventing correct movement of the intended muscle or muscle groups) and attempted gestures (e.g. a hand squeeze or gesture);
   a signal processing module 446 for decoding the brain signals, including extracting electrophysiological features from the brain signals and performing signal processing functions. The extracted features represent brain activities intended for desired actions. In some embodiments, the signal processing module 446 analyzes signal characteristics such as amplitude, latency of event-evoked potentials, frequency power spectra (e.g., sensorimotor rhythms), or neuronal firing rates, in time-domain and/or frequency-domain. In some embodiments, the signal processing module 446 applies mathematical operations to enhance, filter, or modify the signals, allowing for better interpretation and utilization of the data;
   an interpretation module 448 (e.g., decoding module) for decoding the brain signals by recognizing patterns of the extracted features and the intended actions they represent. In some embodiments, the interpretation module 448 implements a classification process to recognize patterns in the extracted features. In some embodiments, the interpretation module 448 applies one or more machine learning models (e.g., data processing models 480 and/or data processing models 568) to decode the brain signals and determine intended actions of the human subject 102. In some embodiments, the interpretation module 448 applies a machine learning algorithm to decode the brain signals and determine intended actions of the human subject 102. In some embodiments, the machine learning algorithm is associated with the one or more machine learning models. In some embodiments, the interpretation module 448 is configured to generate a respective distinct set of intended actions depending on the operating mode (e.g., operating modes 190) of the computing device. In some embodiments, the interpretation module 448 is configured to translate and/or transform the classified features into actual commands;
   an output module 450 for controlling one or more devices, applications, and/or application interfaces to execute the one or more intended actions. In some embodiments, the output module 450 is configured to control the one or more devices, applications, and/or application interfaces to perform dictation control 452, keyboard control 454, cursor control 456, speech control 458, command control 460, accessibility control 462, and alert control 464. In some embodiments, the output module 450 is configured to control an external robotic arm. In some embodiments, the output module 450 is configured to control a prosthetic limb of the human subject 102; and
   a display generation module 466 for generating visual outputs for display on a display device such as a display screen 170 of the computing device 140 of an external display device 173;
one or more other applications 468 that are executed on the computing device 140, Exemplary applications can include a messaging application, an email application, a data presentation and communication application;
a training module 472 for performing limited machine learning model training. In some embodiments, the server system 160 is configured to train one or more machine learning models (e.g., decoding models) that are deployed locally on the computing device 140. In some embodiments, limited model training occurs locally on the computing device 140. In some embodiments, the training module 472 includes:
   one or more training datasets 474; and
   a training task repository 476, which includes training activities or tasks for training the models one or more data processing models 480.
a data store 478 for storing data associated with the computing device 140, including:
   one or more data processing models 480 (e.g., machine learning models, machine learning algorithms, decoding models, and/or decoding algorithms) for supporting the functions performed by the signal processing module 446 and/or the interpretation module 448. As used herein, a data processing model 480 can refer a machine learning model or an algorithm; and
APIs 482 for receiving API calls from one or more applications (e.g., a web browser 426, application 440, other applications 468), translating the API calls into appropriate actions, and performing one or more actions.

Each of the above identified executable modules, applications, or sets of procedures may be stored in one or more of the previously mentioned memory devices, and corresponds to a set of instructions for performing a function described above. The above identified modules or programs (i.e., sets of instructions) need not be implemented as separate software programs, procedures, or modules, and thus various subsets of these modules may be combined or otherwise re-arranged in various embodiments. In some embodiments, the memory 406 stores a subset of the modules and data structures identified above. Furthermore, the memory 406 may store additional modules or data structures not described above. In some embodiments, a subset of the programs, modules, and/or data stored in the memory 406 is stored on and/or executed by a server system 160.

Although FIG. 4 shows a computing device 140, FIG. 4 is intended more as a functional description of the various features that may be present rather than as a structural schematic of the embodiments described herein. In practice, and as recognized by those of ordinary skill in the art, items shown separately could be combined and some items could be separated. In addition, some of the programs, functions, procedures, or data shown above with respect to the computing device 140 may be stored or executed on a server system 160.

FIG. 5 is a block diagram of a server system 160, in accordance with some embodiments. The server system 160 typically includes one or more processors 502 (e.g., processing units or cores (CPUs)), one or more network interfaces 504, memory 506, and one or more communication buses 508 for interconnecting these components (sometimes called a chipset). In some embodiments, the server system 160 includes one or more input devices 510, which facilitate user input, such as a keyboard, a mouse, a voice-command input unit or microphone, a touch screen display, a touch-sensitive input pad, a gesture capturing camera, or other input buttons or controls. In some embodiments, the server system 160 uses a microphone and voice recognition or a camera and gesture recognition to supplement or replace the keyboard. In some implementations, the server system 160 includes one or more cameras, scanners, or photo sensor units for capturing images. In some implementations, the server system 160 includes one or more output devices 512, which enable presentation of user interfaces and display content, including one or more speakers and/or one or more visual displays.

The memory 506 includes high-speed random access memory, such as DRAM, SRAM, DDR RAM, or other random access solid state memory devices. In some implementations, the memory 506 includes non-volatile memory, such as one or more magnetic disk storage devices, one or more optical disk storage devices, one or more flash memory devices, or one or more other non-volatile solid state storage devices. In some implementations, the memory 506 includes one or more storage devices remotely located from the processing units 502. The memory 506, or alternatively the non-volatile memory within the memory 506, includes a non-transitory computer readable storage medium. In some implementations, the memory 506, or the non-transitory computer readable storage medium of the memory 506, stores the following programs, modules, and data structures, or a subset or superset thereof:
an operating system 514, which includes procedures for handling various basic system services and for performing hardware dependent tasks;
a network communication module 516, which connects the server system 160 to other devices (e.g., various servers in the server system 160, computing device 140, a client device, or other devices) via one or more network interfaces 504 (wired or wireless) and one or more networks 150, such as the Internet, other wide area networks, local area networks, metropolitan area networks, and so on;
a user interface module 518, which enables presentation of information (e.g., a graphical user interface for presenting applications, widgets, websites and web pages thereof, and/or games, audio and/or video content) at a computing device 140 or electronic device 178;
a web browser module 520 for navigating, requesting (e.g., via HTTP), and displaying websites and web pages thereof, including a web interface for logging into a user account associated with a computing device 140 or another electronic device, controlling the computing device or electronic device if associated with the user account, and editing and reviewing settings and data that are associated with the user account;
an operational pipeline 524, including:
   a data intake module 526 for obtaining respective neural signals that are collected by respective implant devices;
   a data selection module 528 for automatically choosing the most relevant and suitable data subsets from a larger data pool (e.g., form the one or more databases 162);
   a data processing module 538 for transforming raw sensor data 576 through various processing steps such as data cleaning, normalization, and feature extraction. In some embodiments, the data processing module 538 generates training data for use by a model training module 532 by preparing the processed data in a suitable format;
   the model training module 532 for receiving the training data and establishing one or more data processing models 568 for processing neural data collected by implant devices 110. In some embodiments, the model training module 532 includes labels 534 for training the data processing models 568. In some embodiments, the model training module 532 includes training datasets 536 for training the data processing models 568;
a web application 540, which may be downloaded and executed by a web browser on a user's computing device 140. In general, a web application 540 has the same functionality as application 440, but provides the flexibility of access from any device at any location with network connectivity, and does not require installation and maintenance. In some embodiments, the web application 540 includes various software modules to perform certain tasks, such as:
   a user interface 542, which provides the user interface for all aspects of the web application 540;
   a signal input module 544, which has the same functionalities as signal input module 444
   a signal processing module 546, which has the same functionalities as signal processing module 446;
   an interpretation module 548, which has the same functionalities as interpretation module 448;
   an output module 550, which has the same functionalities as output module 450; and
   a display generation module 552, which has the same functionalities as display generation module 466;
one or more databases 162 for storing at least data including one or more of:
   device settings 562 including common device settings (e.g., service tier, device model, storage capacity, processing capabilities, communication capabilities, etc.) of the one or more server systems 160, computing devices 140, or other devices;
   user account information 564 for the application 440 and/or the web application 540, such as user names, security questions, account history data, user preferences, and predefined account settings
   application data 566 from the application 540, web application 540, and/or the user applications
   firmware data 567 corresponding to respective firmware versions;
   data processing models 568 (e.g., machine learning models, machine learning algorithms, decoding models, and/or decoding algorithms) for decoding neural signal data into intended user actions. In some embodiments, the data processing models 568 include model parameters 570, weights 572, and metadata 574
   raw sensor data 576 acquired by one or more electrode arrays 208;
   derived data 578 obtained by transforming, processing, or analyzing the raw sensor data 576; and
   analytics data 580, which are data that are collected, processed, and analyzed from the raw sensor data 576 and the derived data 578 to gain insights and identify patterns; and
APIs 590 for receiving API calls from one or more applications (e.g., web browser module 520, user applications 522, and web application 540) and the one or more data processing models 568, translating the API calls into appropriate actions, and performing one or more actions.

Each of the above identified executable modules, applications, or sets of procedures may be stored in one or more of the previously mentioned memory devices, and corresponds to a set of instructions for performing a function described above. The above identified modules or programs (i.e., sets of instructions) need not be implemented as separate software programs, procedures, or modules, and thus various subsets of these modules may be combined or otherwise re-arranged in various embodiments. In some embodiments, the memory 506 stores a subset of the modules and data structures identified above. Furthermore, the memory 506 may store additional modules or data structures not described above.

Although FIG. 5 shows a server system 160, FIG. 5 is intended more as a functional description of the various features that may be present rather than as a structural schematic of the embodiments described herein. In practice, and as recognized by those of ordinary skill in the art, items shown separately could be combined and some items could be separated. In addition, some of the programs, functions, procedures, or data shown above with respect to a server system 160 may be stored or executed on a computing device 140. In some embodiments, the functionality and/or data may be allocated between a computing device 140 and one or more server systems 160. Furthermore, one of skill in the art recognizes that FIG. 5 need not represent a single physical device. In some embodiments, the server functionality is allocated across multiple physical devices in a server system. As used herein, references to a “server” include various groups, collections, or arrays of servers that provide the described functionality, and the physical servers need not be physically colocated (e.g., the individual physical devices could be spread throughout the United States or throughout the world).

### Electrode Array Design

As described above, a BCI system can include an array of electrodes coupled to the surface of the sensorimotor cortex of the brain of a human subject to obtain electrical activity on the surface of the brain. The electrodes may be placed exterior to the dura (e.g., epidurally) or interior of the dura (e.g., subdurally). If placed inside the dura, the electrode array is surgically inserted through a slit in the dural and inserted into a cerebrospinal fluid filled space between the brain and the dura. An electrode array that is inserted into the space between the skull and the cortex moves within the space, which may degrade the quality of the measured signals. Accordingly, there is a need for electrode array designs that resist movement and ensure good contact between the electrodes and the surface of the brain.

Various embodiments of the present disclosure are directed to electrode arrays that facilitate accurate measurement of electrical signals on the surface of the brain. As disclosed, in some embodiments the electrode arrays comprise flexible and non-invasive arrays that are positioned on a brain surface (i.e., the electrodes do not penetrate into the brain). Some embodiments are directed to electrode arrays with non-invasive electrodes that are recessed relative to a substrate or recessed under an insulation layer. Some embodiments are directed to electrode arrays with non-invasive electrodes that are flushed with a substrate or ‘proud’ of a lead surface. Unlike existing designs where electrodes are recessed from a lead surface of the lead or recessed under an insulation layer, the disclosed electrode configurations allow electrodes to be closer to the brain for making more intimate contact with the brain surface, thereby improving the quality of the acquired signals. Some embodiments are directed to non-invasive electrode arrays that include a backing layer. In some embodiments, the backing layer is positioned on the back side of the electrode array, opposite the brain surface-facing side of the electrode array where electrodes are located. In some embodiments, the backing layer functions as a gap-filling structure, by filling the subdural space that is normally filled with cerebrospinal fluid. The backing layer facilitates a slight downward pressure on the electrode array and improves electrode contact with the brain. For example, the backing layer functions can keep the electrodes in contact with the brain during brain pulsation (e.g., due to respiratory/cardiac cycles) or other brain motion. In some embodiments, a portion of the flexible electrode is curved with a radius of curvature that approximates a curvature of the brain. The radius of curvature is selected so that the flexible electrode creates a downward force that keeps the electrodes in contact with the brain during brain pulsation. In some embodiments, the combination of the curved portion of the flexible electrode and the backing layer facilitates a slight downward pressure on the electrode array and improves electrode contact with the brain. In some embodiments, a radius of the curvature of the flexible substrate and/or a combined thickness of the flexible substrate and the backing layer, are selected or adapted to provide sufficient pressure on the array to improve contact with the brain and at the same time not apply over pressure on the brain. In some embodiments, the backing layer reduces the space between the dura and the brain thus preventing or limiting movement of the electrode array relative to the brain surface. By improving contact with the brain, better quality neural signals can be collected.

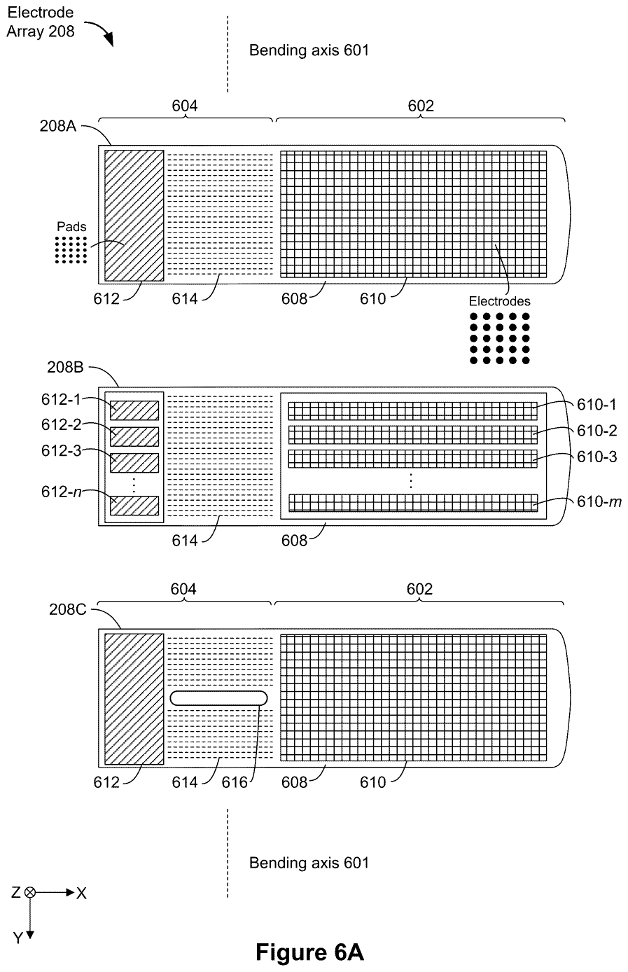

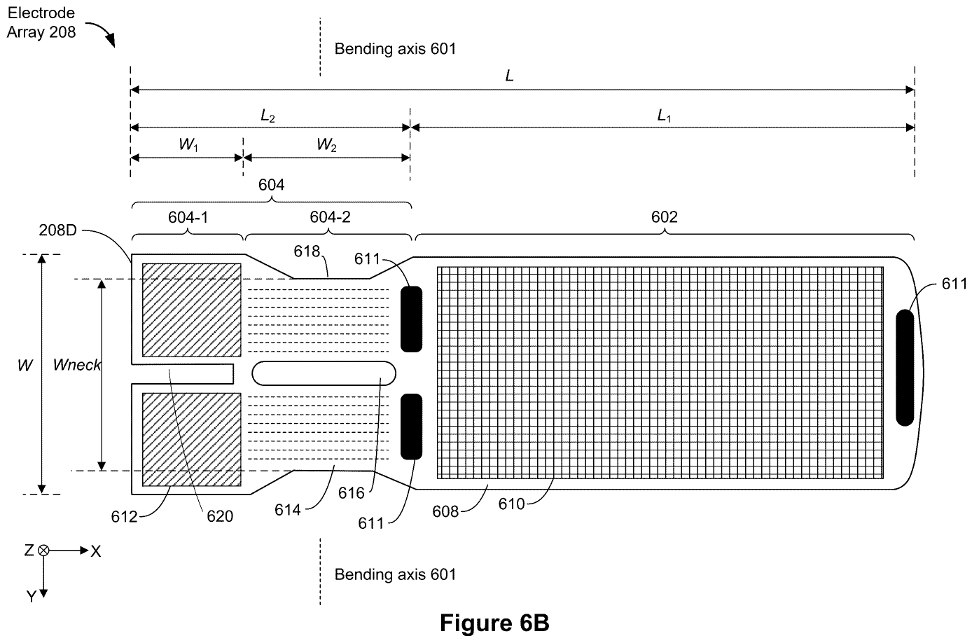

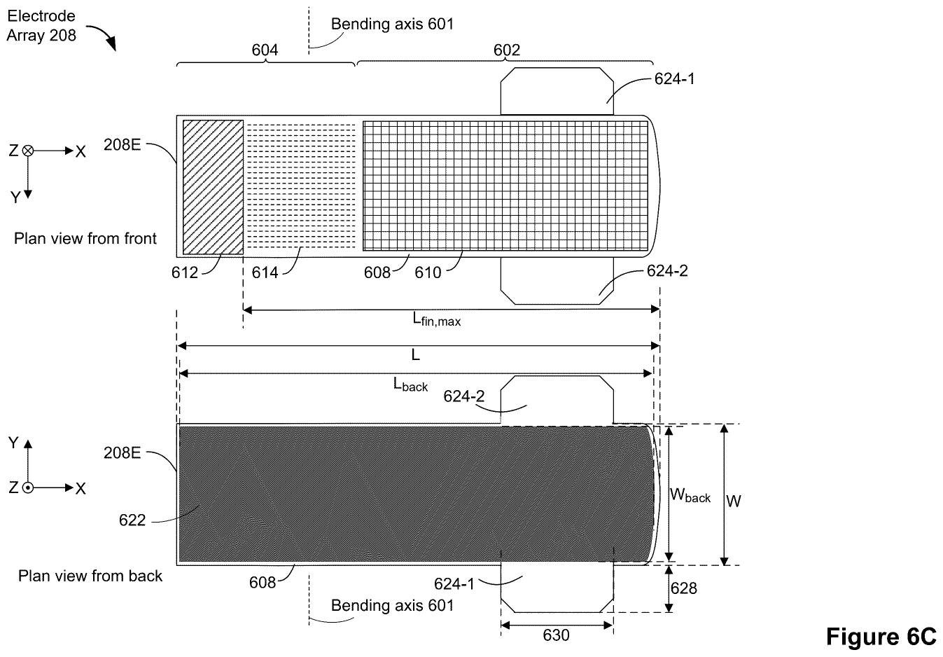

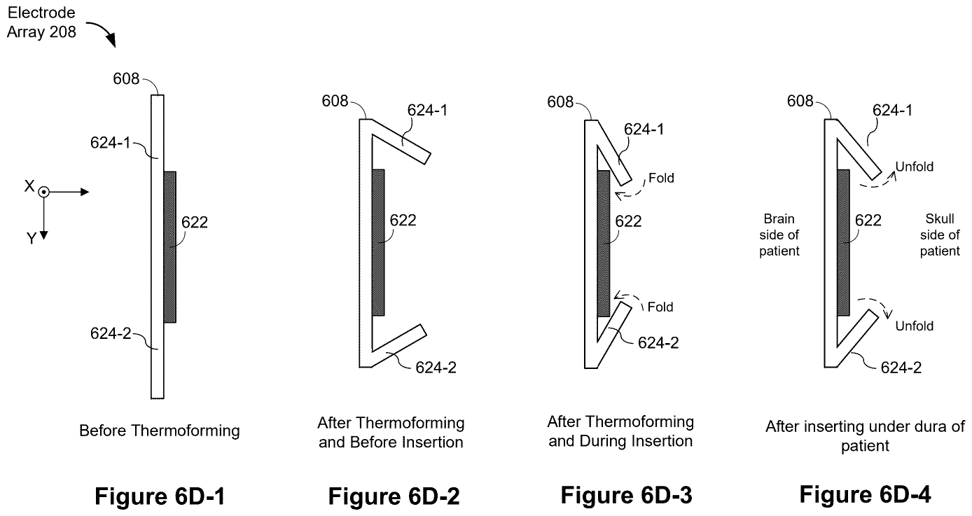

FIGS. 6A to 6C illustrate example electrode arrays 208A to 208E of the implant device 110, in accordance with some embodiments. The electrode arrays 208A to 208E are similar to the electrode array 208 shown in FIGS. 2A-2C, but differ in terms of (1) geometric configurations and (2) distribution of electrodes and/or bond pads mounted thereon. Any of the electrode arrays 208A to 208E can be coupled to the frame 202 of the implant device 110. In some embodiments, the electrode array 208 is surgically positioned on a brain surface of a patient. In some embodiments, the electrode array 208 is configured to be positioned on the brain surface of the patient epidurally, i.e., above a dura mater layer. Alternatively, in some embodiments, the electrode array 208 is configured to be positioned on the brain surface of the patient subdurally, under the dura mater layer.

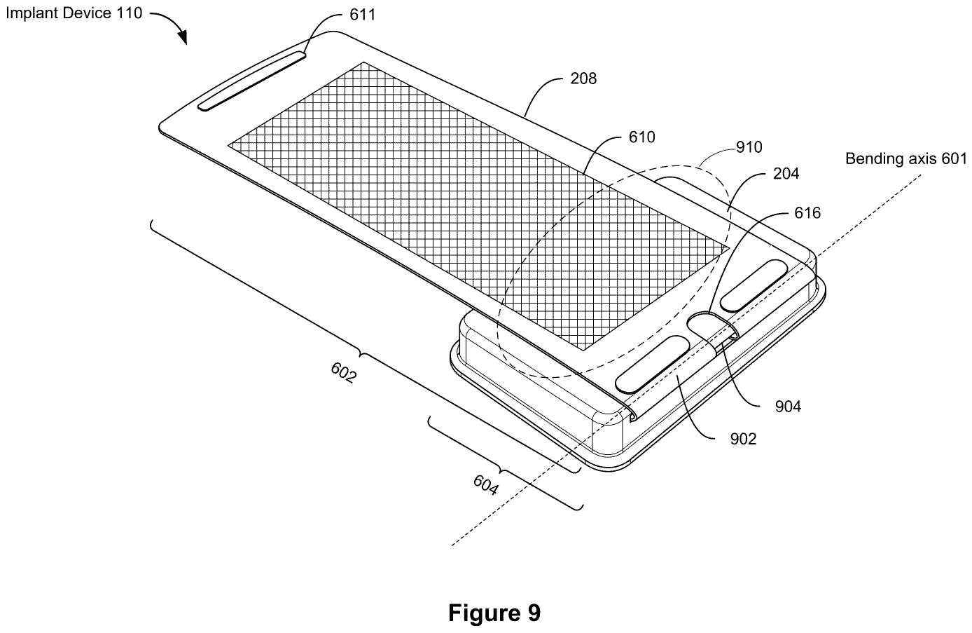

FIG. 6A illustrates electrode arrays 208A to 208C with different configurations. Electrode array 208A includes a first portion 602 and a second portion 604 that are contiguous to each other. The electrode array 208A further includes a bending axis 601 (e.g., Y axis, along the width of the electrode array). In some embodiments, a bent portion (e.g., discussed below in FIGS. 9-11) is formed by bending the first portion 602 of the electrode array 208A with respect to the second portion 604 of the electrode array 208A at the bending axis 601 along a width (e.g., denoted by W in FIG. 6B) of the electrode array 208A.

In some embodiments, the electrode array 208 includes a flexible substrate 608. In some embodiments, the flexible substrate 608 is stiff enough to be inserted into the narrow space between the skull and the brain of the patient, and yet flexible enough to conform to the curved space between the brain and the skull.

In some embodiments, the flexible substrate 608 has a thickness of about 50 μm to 200 μm. For example, in some embodiments, the thickness of the flexible substrate is in the range of about 50 μm to 100 μm, 50 μm to 150 μm, 50 μm to 200 μm, 100 μm to 150 μm, 100 μm to 200 μm, or 100 μm to 200 μm.

In some embodiments, the flexible substrate 608 comprises a biocompatible material. In some embodiments, the flexible substrate 608 includes a biocompatible liquid crystal polymer (LCP) material that is fabricated as a thin film with mechanical robustness and thermal stability. In some embodiments, the LCP material of the flexible substrate 608 includes molecules with rigid and flexible monomeric units. The rigid monomeric units provide high temperature capability and good mechanical properties, while the flexible monomeric units provide the processability of the molecule chains. In some embodiments, the flexible substrate comprises a polymer material such as polyimide (PI), polydimethylsiloxane (PDMS), and parylene C.

In some embodiments, the electrode array 208 includes a plurality of electrodes 610 (e.g., electrode sensors 210 in FIG. 2A) positioned at the first portion 602. The electrodes 610 are mounted on and protrude from the flexible substrate 608. In some embodiments, the electrodes 610 are deposited on a surface of the flexible substrate 608.

FIG. 6A shows that in some embodiments, the electrode array 208 includes a plurality of bond pads 612 that are mounted on the flexible substrate 608 and positioned at the second portion 604. The bond pads 612 are electrically coupled to a set of electronic components (e.g., implant electronics 206) of the implant device 110. In some embodiments, the flexible substrate 608 includes electrode leads 614 that electrically couple the electrodes 610 to the bond pads 612. In some embodiments, the electrode leads 614 are embedded within the flexible substrate 608. In some embodiments, the bond pads 612 are at least equal in number to the electrodes 610. In some embodiments, the bond pads 612 are at least equal in number to the electrode leads 614.

In some embodiments, at least some of the plurality of electrodes 610 are configured to be in physical contact with the brain surface of the patient when positioned in the patient. In some embodiments, at least some of the plurality of electrodes 610 are configured to be proximate to (e.g., placed just above) a dural layer of the brain of the patient. For example, in some embodiments, the at least some of the plurality of electrodes 610 are configured to be within 1 micrometers (μm), within 5 μm, within 10 μm, within 0.1 mm, within 0.2 mm, or within 0.5 mm of the brain surface of the patient.

The electrodes 610 are made of a biocompatible material. Exemplary biocompatible materials include, and are not limited to, platinum (Pt), titanium (Ti), gold (Au), tantalum (Ta), certain stainless steel materials, or carbon-based materials such as carbon nanotubes and carbon nanofibers.

FIG. 6A shows an electrode array 208B, which is similar to electrode array 208A, but differs primarily in that the plurality of electrodes 610 and/or the plurality of bond pads 612 of the electrode array 208B are arranged in groups. In some embodiments, arranging the electrodes 610 and/or the bond pads 612 in groups may offer several advantages, including (1) enhanced flexibility in electrical connections between the plurality of electrodes 610 and the plurality of bond pads 612 through the electrode leads 614, (2) optimized detection sensitivity for acquiring neural signals (e.g., electrical signals) with reduced electrical interference, and (3) increased design flexibility in shaping the geometry of the electrode array 208B to improve mechanical robustness (e.g., discussed below in FIG. 6B). In some embodiments, the plurality of electrodes 610 includes one or more groups of electrodes (e.g., group 610-1 to group 610-m, where m is an integer greater than one). In some embodiments, the plurality of bond pads 612 includes one or more groups of bond pads (e.g., group 612-1 to group 612-n, where n is an integer greater than one).

FIG. 6A also shows an electrode array 208C, which is similar to the electrode array 208A, but differs primarily in that the electrode array 208C includes an opening 616 located at the second portion 604 of the electrode array 208C. In some embodiments, the opening 616 is positioned between the first portion 602 and the second portion 604. In some embodiments, the opening 616 is positioned at the first portion 602. In some embodiments, the opening 616 comprises a void space. In some embodiments, the opening 616 is a void space. In some embodiments, the opening 616 includes a mesh material or a bioabsorbable material.

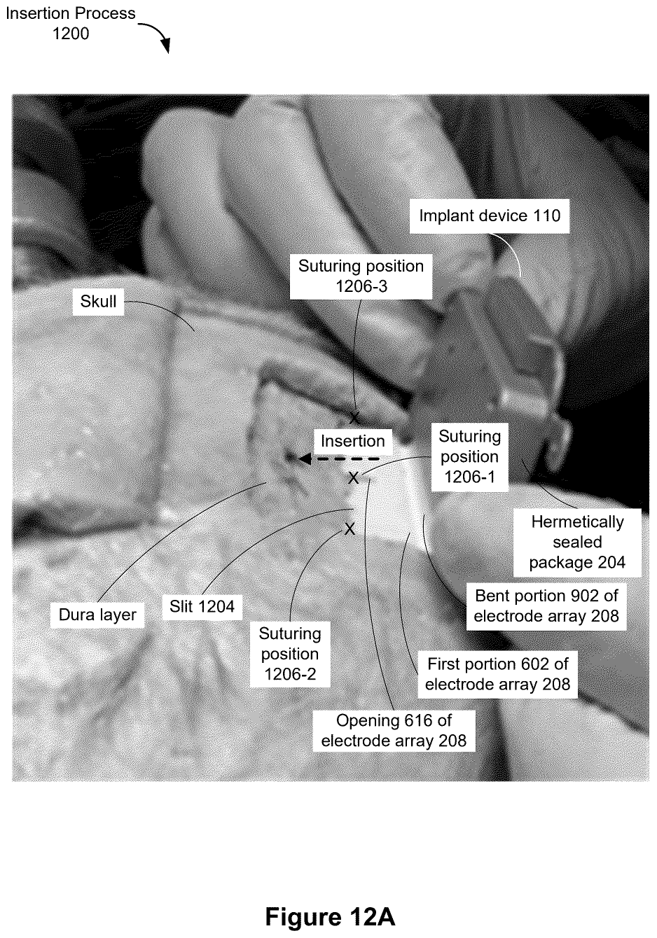

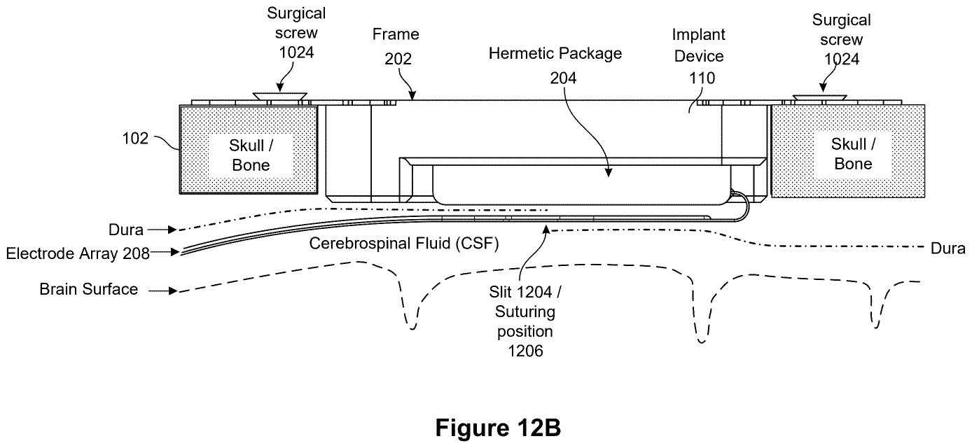

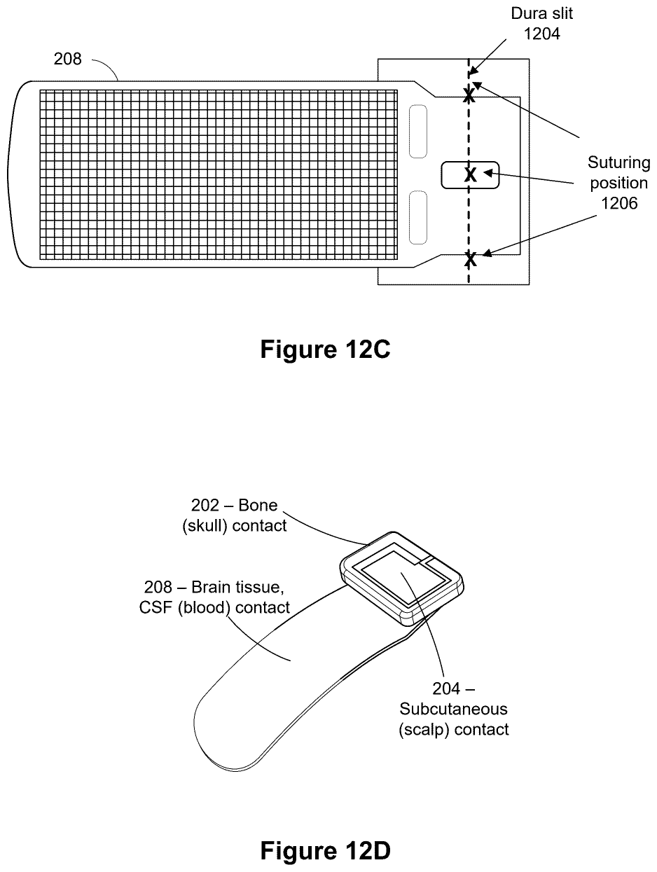

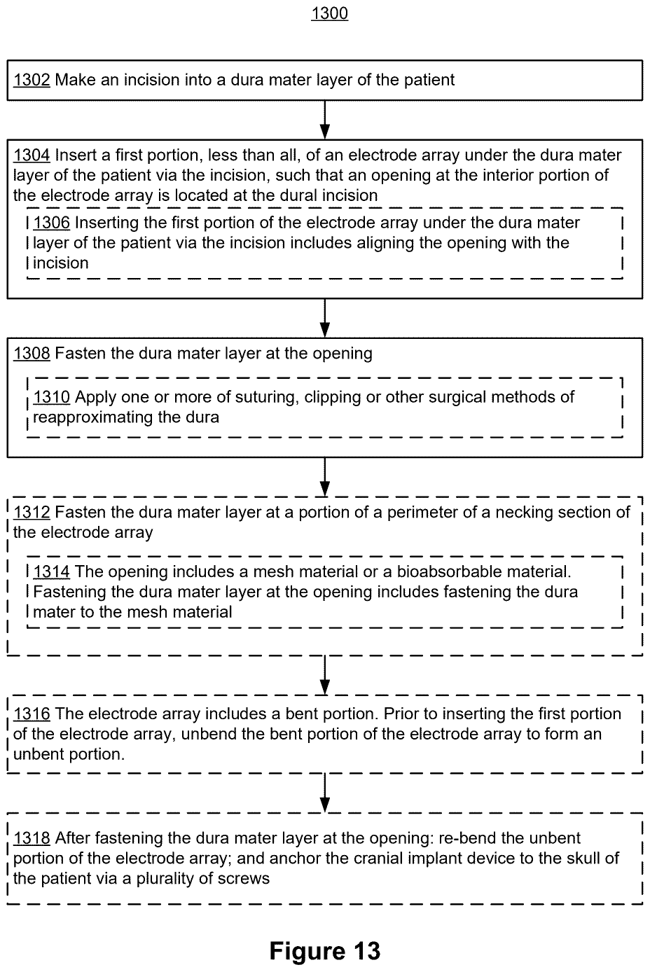

In accordance with some embodiments, the implant device 110 (including at least a portion of the electrode array 208) is configured to be inserted into the skull of a patient via a craniectomy process. The process includes making an incision (e.g., a slit, such as slit 1204 in FIG. 12) on the dura mater layer and inserting a portion of the electrode array through the slit, into the dura mater layer. In some embodiments, the opening 616 is configured to align with the slit (see FIG. 12) and enables the electrode array 208 to be sutured to the dura (e.g., at suturing position 1206-1 in FIG. 12).

FIG. 6B shows an electrode array 208D in accordance with some embodiments. In FIG. 6B, the second portion 604 includes a first section 604-1 and a second section 604-2 contiguous to the first section 604-1. In this example, the second section 604-2 corresponds to a necking section 618 on both sides of the electrode array 208D, where a width W_{neck} of the necking section 618 is smaller than the width W of the electrode array 208. The electrode array 208D includes the opening 616 positioned at the second section 604-2. In this configuration, the opening 616 combined with the smaller width of the necking section 618 provides additional regions for suturing the electrode array 208D onto the dural layer of the patient during the craniectomy process as described above, thereby securing the placement of the implant device 110 on the brain surface of the patient.

In some embodiments, the electrode array 208D includes one or more electrode pads 611 (e.g., positioned at the first portion 602). In some embodiments, the one or more electrode pads 611, together with the plurality of electrodes 610, facilitate acquisition of neural signals. In some embodiments, a respective electrode pad 611 is larger than a respective electrode 610.

In some embodiments, the electrode array 208D includes a cutout region 620. In some embodiments, the cutout region 620 provides mechanical robustness for integrating the electrode array 208B into the hermetic package 204.

In some embodiments, the electrode array 208 has a length L and a width W. In some embodiments, the length L is about 110 millimeters (mm) to 150 mm. For example, in some embodiments, the length L has a range of values of around 110 mm to 120 mm, 110 mm to 130 mm, 110 mm to 140 mm, 110 mm to 150 mm, 120 mm to 130 mm, 120 mm to 140 mm, 120 mm to 150 mm, 130 mm to 140 mm, or 130 mm to 150 mm.

In some embodiments, the width W is about 30 mm to 40 mm. For example, in some embodiments, the width W has a range of values of around 30 mm to 32 mm, 30 mm to 35 mm, 30 mm to 38 mm, 30 mm to 40 mm, 32 mm to 36 mm, 32 mm to 38 mm, 32 mm to 40 mm, 34 mm to 36 mm, 34 mm to 38 mm, 34 mm to 40 mm, 36 to 38 mm, or 38 to 40 mm.

In some embodiments, the first portion 602 of the electrode array 208 has a first length L₁. In some embodiments, the first length L₁ of the first portion 602 is about 70 mm to 80 mm. In some embodiments, the first length L₁ of the first section 604-1 has a range of values of around 70 mm to 74 mm, 70 mm to 77 mm, 70 mm to 80 mm, 72 mm to 74 mm, 72 mm to 77 mm, 72 mm to 80 mm, 75 mm to 77 mm, or 75 mm to 80 mm.

In some embodiments, the second portion 604 of the electrode array 208 has a second length L₂. Note that the total length L is equal to the sum of the first length L₁ of the first portion 602 and the second length L₂ of the second portion 604.

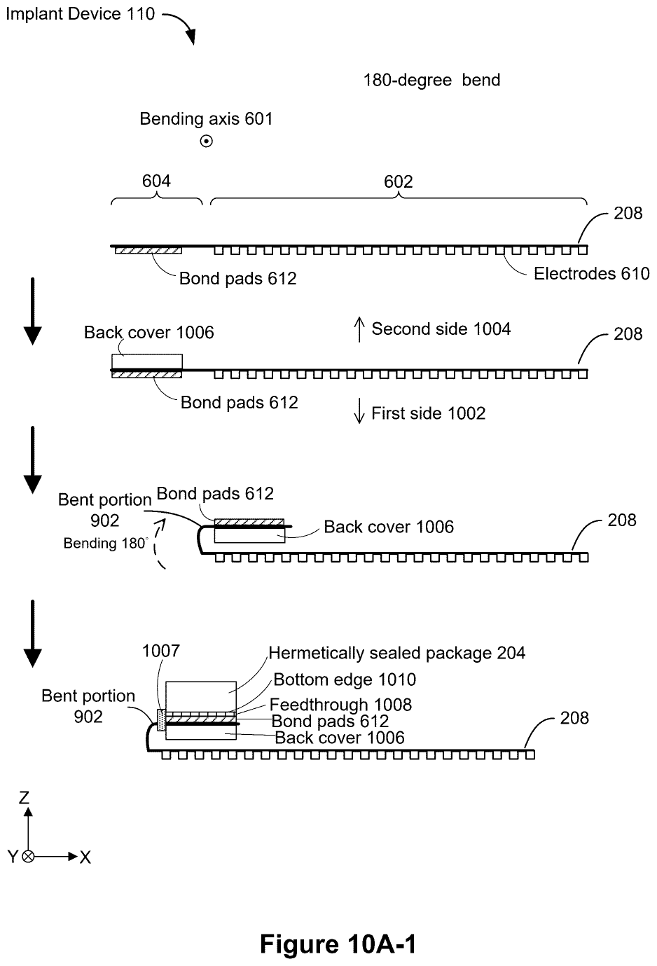

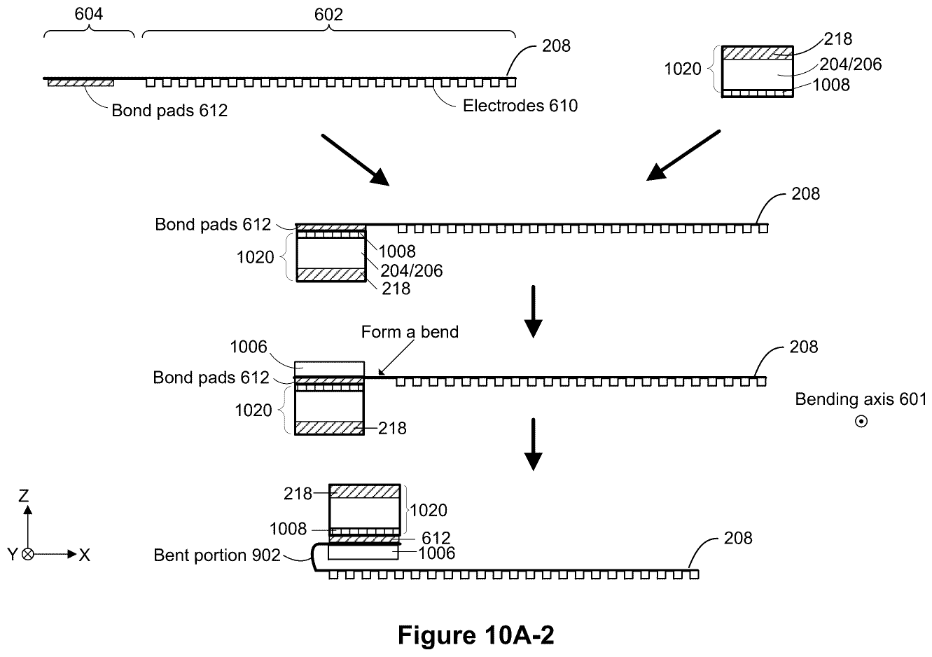

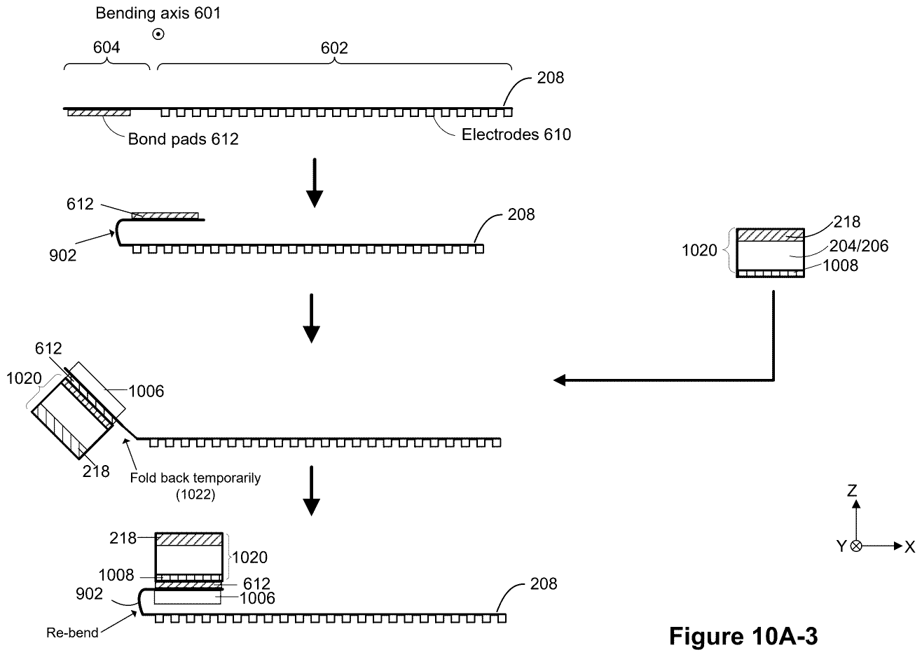

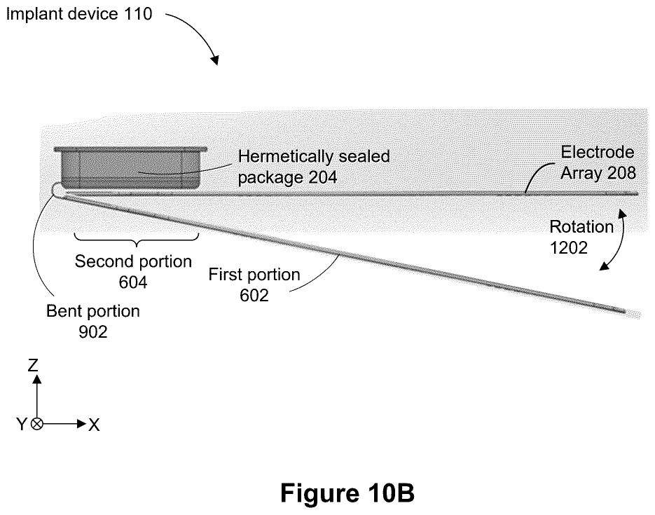

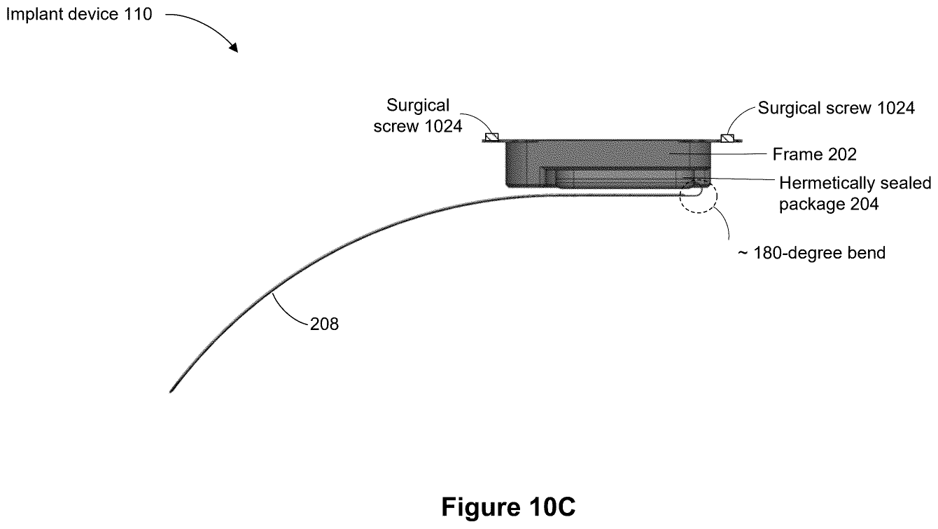

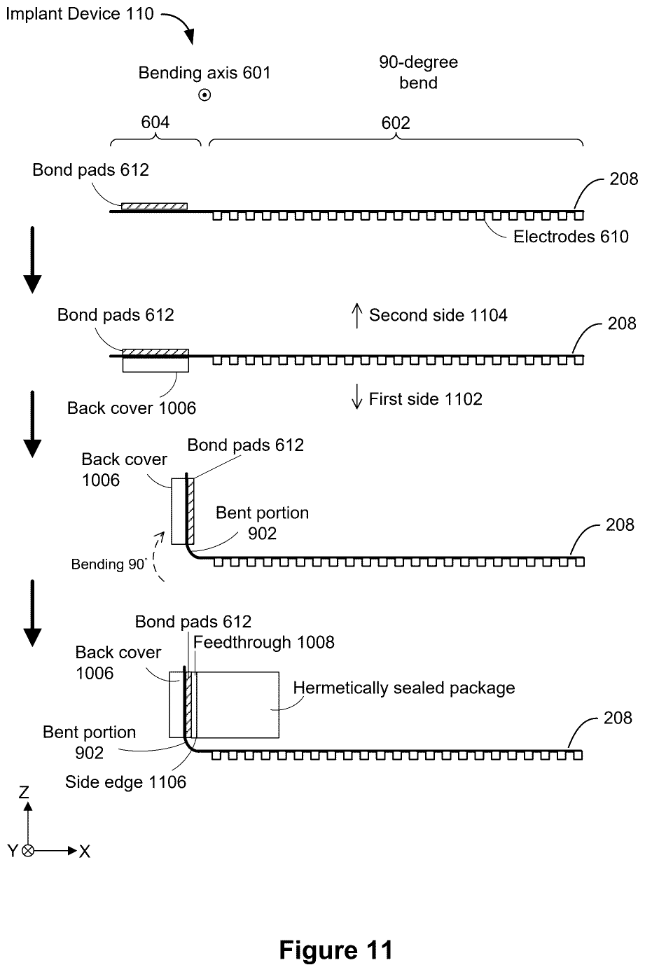

In some embodiments, the first section 604-1 of the second portion 604 of the electrode array 208 has a first width W₁ and the second section 604-2 of the second portion 604 of the electrode array 208 has a second width W₂. In some embodiments, the first width W₁ is around 20 mm to 50 mm. In some embodiments, the second width W₂ is around 20 mm to 50 mm. In some embodiments, the second width W₂ is smaller than the first width W₁. In some embodiments, the second width W₂ is equal to the first width W₁. In some embodiments, the second width W₂ is greater than the first width W₁. In some embodiments, the first width W₁ is determined by various factors, such as bending configurations (e.g., 180-degree bend and 90-degree bend in FIGS. 10 and 11) and thickness of the electrode array 208. In some embodiments, the second width W₂ is determined by various factors, such as size of the opening 616 and suturing requirement.

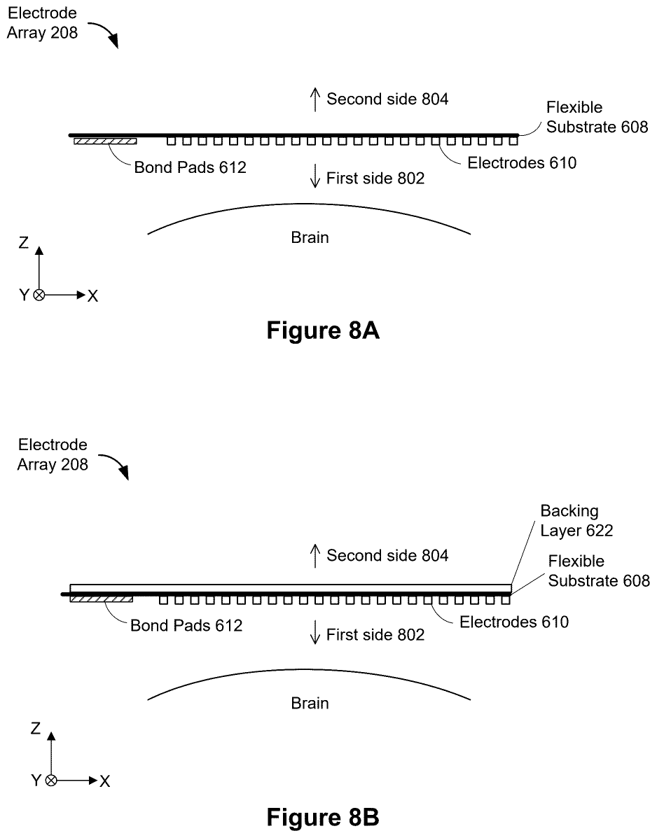

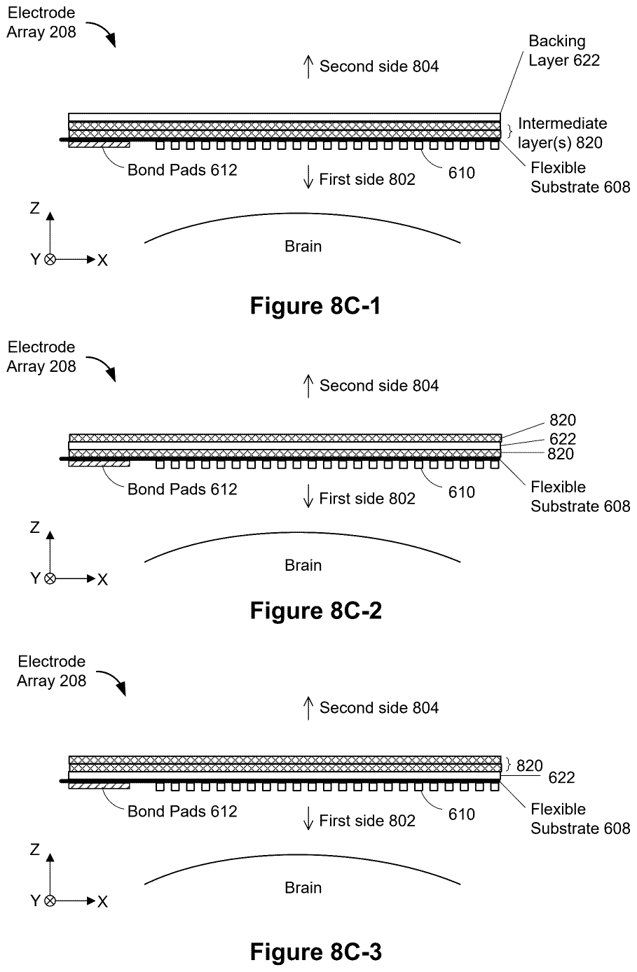

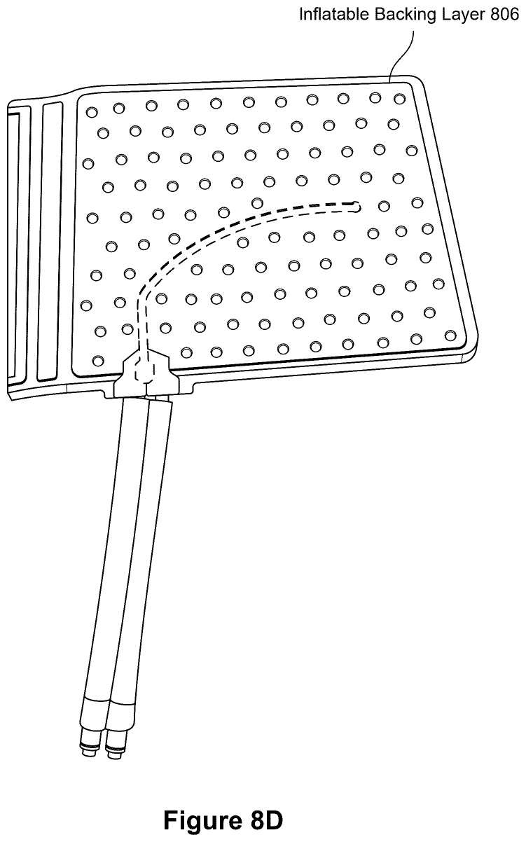

FIG. 6C illustrates an electrode array 208E that includes a backing layer 622. In this example, the backing layer 622 is disposed on a back side (e.g., second side 804 in FIG. 8B) of the electrode array 208E. In accordance with some embodiments, the backing layer 622 acts as a gap filler by reducing a space between the dural layer and the brain of the patient so as to prevent or limit movement of the electrode array 208. By limiting movement of the electrode array 208 relative to the brain surface, electrode contact is improved, which can in turn lead to improved quality of the collected neural signals. In some embodiments, the backing layer 622 is configured to hold the electrode array 208 in-place when the electrode array is inserted subdurally. In some embodiments, the backing layer 622 has a length L_{back} that is substantially similar to the length L of the electrode array 208 (e.g., exactly the same, or within ±1%, ±2%, ±5% of each other). In some embodiments, the backing layer 622 has a width W_{back} that is substantially similar to the width W of the electrode array 208 (e.g., exactly the same, or within ±1%, ±2%, ±5% of each other). In some embodiments, the size of the backing layer 622 is smaller (e.g., slightly smaller) than the size of the electrode array 208E.

FIG. 6C depicts that in some embodiments, the flexible substrate 608 includes one or more fins 624 (e.g., fin 624-1 and fin 624-2) (e.g., also referred to as flaps or wings). In particular, the one or more fins 624 extend from one or more respective edges (e.g., long edges) of the flexible substrate 608, in a width direction of the flexible substrate 608 (e.g., in the Y direction). In the example of FIG. 6C, a respective fin 624 extends by a first dimension 628 from the long edge of the flexible substrate 608. FIG. 6C shows that the respective fin 624 can extend for a portion 630 (e.g., a dimension) of the total length L of the flexible substrate 608. In some embodiments, the portion 630 can be at least 10%, 20%, 30%, 40%, 50%, 60%, 70%, 80%, or 90% of the total length L of the flexible substrate 608. In some embodiments, the maximum length of the portion 630 is equal to the length of the flexible substrate that is occupied by the electrodes 610 and the electrode leads 614. This is denoted by L_{fin,max} in FIG. 6C.

In some embodiments, the one or more fins 624 are configured to be bent (e.g., folded) along the one or more respective edges (e.g., long edges) of the flexible substrate 608, toward the back side of the flexible substrate 608 (e.g., the side where the backing layer 622 is positioned), via a thermoforming process that heats the fins 624 and forms them (e.g., over a mold) using vacuum, pressure, or mechanical force. FIGS. 6D-1 to 6D-4 are schematic side views of the electrode array 208E illustrating the flexible substrate 608 with the fins 624 (i) before the thermoforming process, (ii) after the thermoforming process and before the flexible electrode array 208E is inserted into a patient, (iii) after the thermoforming process and during insertion of the flexible electrode array 208E under the patient's dura, and (iv) after the flexible electrode 208E has been inserted under the patient's dura, respectively. The fins 624 are configured to fold toward the backing layer 622 (e.g., approximately flat) during insertion, as illustrated in FIG. 6D-3, and configured to unfold distally toward the skull of the patient after the portion of the electrode array has been inserted, as illustrated in FIG. 6D-4, thereby holding the electrode array 208E (e.g., electrodes 610) against the brain surface of the patient. The angles of the fins 624 after unfolding (e.g., FIG. 6D-4) may be different from the angles of the fins 624 after thermoforming and before insertion (e.g., FIG. 6D-2).

In some embodiments, the one or more fins 624 are configured to be bent using a thermoforming process.

In some embodiments, the one or more fins 624 are made of the same material as the backing layer 622. For example, the backing layer 622 and the one or more fins 624 are both made of silicone. In some embodiments, the one or more fins 624 are made of a material different from that of the backing layer 622. For example, the one or more fins 624 is made of LCP whereas the backing layer 622 is made of silicone. In some embodiments, after the portion of the electrode array has been inserted and the fins 624 unfolded, one or more surfaces 626 of the fins 624 may physically contact the dural layer of the patient. In some embodiments, prior to electrode insertion, a silicone coating can be applied to surfaces 626 of the fins 624 that would likely contact the dural layer of the patient once inserted.

FIGS. 8A to 8D illustrate various embodiments of the backing layer 622 and placement of the electrode array 208 relative to the brain surface of the patient, in accordance with some embodiments. As shown in FIG. 8A, the electrode array 208 (e.g., electrode arrays 208A to 208E in FIGS. 6A-6C) includes the flexible substrate 608 with the electrode leads 614, the plurality of electrodes 610, and the plurality of bond pads 612. In particular, the plurality of electrodes 610 and the plurality of bond pads 612 are electrically coupled through the electrode leads 614.

Turning to FIG. 8B, the electrode array 208 includes the backing layer 622. Electrodes 610 are mounted on a first side 802 of the flexible substrate 208 and are configured to be positioned on the brain surface of the patient. The flexible substrate 208 includes a second side 804, opposite to the first side 802, that is coupled to the backing layer 622. In some embodiments, the backing layer 622 acts as a gap filler between the dura layer and the brain of the patient to prevent relative movement of the flexible substrate 608 on the brain surface. In some embodiments, one or more intermediate layers 820 may also be coupled with the flexible substrate 608, as illustrated in various arrangements depicted in FIGS. 8C-1 to 8C-3. For example, FIG. 8C-1 shows the second side of the flexible substrate is coupled to a respective intermediate layer 820 of the one or more intermediate layers, and the backing layer is coupled to a respective intermediate layer 820. FIG. 8C-2 shows the backing layer 622 sandwiched between two intermediate layers 820. FIG. 8C shows the backing layer 622 is coupled to the second side of the flexible substrate, a respective intermediate layer 820 of the one or more intermediate layers is coupled to the backing layer.

In some embodiments, the backing layer 622 comprises a silicone backing layer. In accordance with some embodiments, attaching a silicone backing layer to the electrode array 208 can not only hold the electrode array in place when the electrode array is inserted subdurally (e.g., by reducing the space between the dura layer and the surface of the patient's brain), but also increases the overall stiffness of the electrode array 208 to guide the array under the dura during the surgical process, thereby facilitating surgical insertion. In some embodiments, the backing layer 622 that includes the silicone backing layer has a thickness of about 250 μm to 300 μm, 250 μm to 400 μm, or 250 μm to 500 μm, which corresponds to roughly the gap space between the dura layer and the brain surface.

In some embodiments, the backing layer 622 has a thickness of about 0.25 mm to 1 mm. In some embodiments, the thickness of the backing layer 622 is adjustable (e.g., variable) according to an anatomy of the patient. In some embodiments, the thickness of the backing layer is adjustable (e.g., variable) according to a respective region of the brain in which the implant device 110 is configured to be positioned. For example, when the backing layer 622 is too thin, the space between the dura layer (e.g., a respective region of the dura layer) and the brain (e.g., a respective region of the brain) is not reduced, which can lead to movement of the electrode array 208. On the other hand, when the backing layer 622 is too thick, the electrode array 208E may exert additional pressure on the dural layer and/or the brain of the patient, which is also not desirable.

In some embodiments, the backing layer 622 includes an inflatable backing layer that is adjustable for each patient, e.g., based on the respective spacing between the dural layer and the brain of the patient. FIG. 8D illustrates an inflatable backing layer 806 in accordance with some embodiments. In some embodiments, the backing layer 622 (or inflatable backing layer 806) includes a cavity (e.g., internal space) that is filled with one or more materials, including one or more of: a flowable silicone elastomer, a plastic bead, a mineral oil, a saline solution, or a hydrogel. Such materials may be selected and introduced into the cavity to provide an inflatable or otherwise conformable backing structure having a thickness that can better match a local spacing between the dura and the brain. By better matching the local spacing, the backing layer can improve fit between the electrode array and surrounding anatomy, reduce a likelihood that the electrode array moves due to an insufficient backing thickness, and reduce a likelihood that the electrode array exerts undesirable pressure on the brain due to an excessive backing thickness. In some embodiments, the backing layer 622 (or inflatable backing layer 806) includes a compressible material such as foams, sponges, rubber, or other compressible materials with a porous or flexible structure. In some embodiments, a thickness of the inflatable backing layer is adjustable so the backing layer can better match the local spacing between the dura and the brain. Consequently, this improves fit, reduces the risk of the array moving due to the layer being too thin, and reduces the risk of the array exerting undesirable pressure on the brain of the patient due to the layer being too thick.

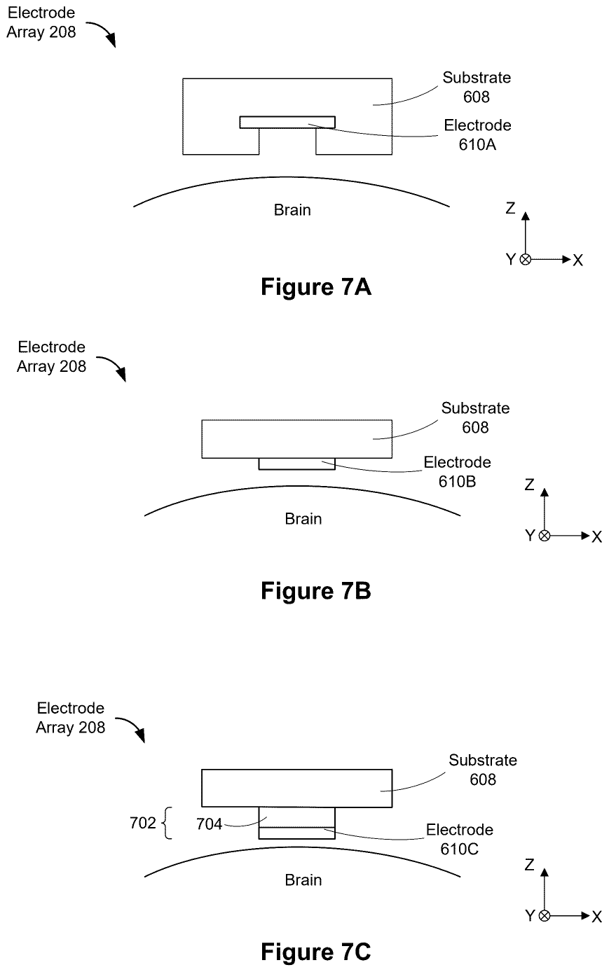

FIGS. 7A to 7C illustrate example electrode configurations implemented in the electrode array 208 (e.g., electrode arrays 208A to 208E), in accordance with some embodiments. As shown in FIG. 7A, a plurality of electrodes 610A (e.g., electrodes 210 and 610) are recessed relative to the surface of the flexible substrate 608 to form recessed electrodes. In particular, the recessed configuration of the plurality of electrodes 610A prevents tissue scarring on the brain of the patient, though it may reduce signal accuracy due to (i) movement of the electrode array 208 and (ii) lack of proximity to the brain surface. Turning to FIG. 7B, a plurality of electrodes 610B (e.g., similar to electrodes 210 and 610) are deposited on the surface of the flexible substrate 608 to form flush electrodes. Compared with the recessed electrodes 610A, this flush configuration enables more intimate electrical contacts with the brain surface, which can in turn improve the quality of the neural (e.g., electrical) signal acquisition.

Turning to FIG. 7C, a plurality of electrodes 610C (e.g., electrodes 210 and 610) are deposited on the protruded surface of the flexible substrate 608, e.g., proud electrodes. In particular, the electrode array 208 includes an additional layer 702 that is applied to the flexible substrate 608 and stands proud of the flexible substrate 608, where the additional layer 702 includes a substrate portion 704 and a sensor layer (e.g., as electrodes 610C), such that the plurality of electrodes 610C stand proud of the additional layer 702.

### Flexible Connector Design

Various embodiments are directed to implant devices with flexible connectors. In particular, the disclosed implant device is configured to maximize the coverage of a sensorimotor cortex region of a patient while minimizing the total footprint of the implant device. In some embodiments, the implant device includes electronic components that are contained in a hermetically sealed package and a flexible electrode array. By routing electrodes of the flexible electrode array underneath the hermetically sealed package, the implant device reduces its overall footprint, thereby reducing the area of a skull required to be removed during craniectomy. Positioning the electrodes (or a subset thereof) underneath the hermetically sealed package allows neural signals that are directly under the hermetically sealed package to be collected.

In accordance with some embodiments, the implant device is configured to be implanted into the skull near the top of the patient's head, where the skull is not covered by muscle. Thus, the craniectomy process can be performed without the need to adjust or move muscles around the patient's skull.

In accordance with some embodiments, the flexible electrode array is configured to be positioned on the brain surface of the patient. Accordingly, the implant device is less invasive compared to other implant devices with electrodes that penetrate into the brain.

FIG. 9 illustrates an example implant device 110 including a hermetically sealed package 204 and a bent electrode array 208 featuring a flexible connector, in accordance with some embodiments. In some embodiments, the implant device 110 includes a hermetically sealed package 204 (e.g., an “electronics can” similar to the hermetic package 204 in FIGS. 2A to 2C) configured to be inserted into an opening in a skull of a patient, implant electronics 206 (e.g., electronic components 206) enclosed within the hermetically sealed package 204, and a flexible electrode array (e.g., electrode array 208) configured to be positioned on a brain surface of the patient. In some embodiments, the flexible electrode array 208 includes the first portion 602 and the second portion 604. The second portion 604 is not fully visible in FIG. 9. In some embodiments, the flexible electrode array 208 includes the plurality of electrodes 610 mounted on the first portion 602 and the plurality of bond pads 612 mounted on the second portion 604. The electrodes 610 and the bond pads 612 are electrically coupled through electrode leads (e.g., electrode leads 614 in FIGS. 6A-6C). In some embodiments, the flexible electrode array 208 includes the one or more electrode pads 611 that facilitate acquisition of neural signals (e.g., electrical signals). In some embodiments, the flexible electrode array 208 includes the opening 616 for suturing during a surgical insertion of the implant device 110.

In some embodiments, the flexible electrode array 208 includes a bent portion 902 that is configured to be coupled (e.g., attached) to an edge 904 (e.g., a side edge or a bottom edge) of the hermetically sealed package 204 such that a subset of electrodes 910 of the flexible electrode array 208 is positioned under the hermetically sealed package 204. The bent portion 902 is formed by bending the first portion 602 with respect to the second portion 604 at the bending axis 601 along a width W of the flexible electrode array 208, as illustrated in FIGS. 6A to 6C. Bending the first portion 602 of the electrode array 208 with respect to the second portion 604 of the electrode array 208 enables the second portion 604 of the flexible electrode array 208 to electrically couple to the hermetically sealed package 204 via a hermetic feedthrough 1008 (e.g., an electrical feedthrough) of the hermetically sealed package 204. The flexible electrode array 208 is electrically connected to the implant electronics 206 via electrode leads 614 and bond pads 612, then via the hermetic feedthrough 1008, and then to the implement electronics 206. In some embodiments, the bent portion 902 is the portion of the flexible electrode array 208 that includes bond pads (e.g., bond pads 612 in FIGS. 6A-6C). In some embodiments, the bent portion 902 at least a part of the second portion 604 of the flexible electrode array 208.

FIG. 10A-1 illustrates an example process of forming the bent portion 902 of the flexible electrode array 208, featuring a 180-degree bend, in accordance with some embodiments. In some embodiments, the flexible electrode array 208 includes electrodes 610 and the bond pads 612 formed on a first side 1002 of the flexible electrode array 208. A back cover 1006 is positioned on a second side 1004 of the flexible electrode array 208 and coupled to the plurality of bond pads 612 through the second portion 604 of the flexible electrode array 208. In some embodiments, the bent portion 902 of the flexible electrode array 208 is formed by bending the first portion 602 of the flexible electrode array 208 with respect to the second portion 604 of the flexible electrode array 208 at the bending axis 601 (e.g., Y axis) along a width W of the flexible electrode array 208. In some embodiments, the first portion 602 of the flexible electrode array 208 is contiguous to the second portion 604 of the flexible electrode array 208. In some embodiments, the bent portion 902 is formed using a thermoforming process.

In some embodiments, the bent portion 902 of the flexible electrode array 208 is coupled to a bottom edge 1010 (e.g., a long edge) of the hermetically sealed package 204. The bent portion 902 of the flexible electrode array 208 is configured to couple to the bottom edge 1010 of the hermetically sealed package 204 via a hermetic feedthrough 1008 (e.g., an electrical feedthrough). In some embodiments, the bent portion 902 of the flexible electrode array 208 is configured to couple to the bottom edge 1010 of the hermetically sealed package 204 via the hermetic feedthrough 1008 at the first side 1002 of the bent portion 902 and configured to couple to the back cover 1006 at the second side 1004 of the bent portion 902. In some embodiments, the hermetic feedthrough 1008 is part of the hermetically sealed package 204 and configured to electrically connect the set of electronic components 206 to the flexible electrode array 208. In some embodiments, the back cover 1006 is part of the implant device 110. In some embodiments, a layer of material 1007 is placed on a surface of the flexible electrode array where the flexible electrode array meets a frame. For example, the layer of material 1007 may be placed between the flexible electrode array 208 and the back cover 1006. In some embodiments, the layer of material 1007 is a silicone layer. The function of the layer 1007 is to create a transition or strain relief region that ensures system reliability by managing mechanical stress, sealing against environmental exposure, and maintaining electrical signal integrity (e.g., similar to a cable connector).

In some embodiments, the 180-degree flex (e.g., bending angle) as illustrated in FIG. 10A-1 allows for movement of the flexible electrode array 208 without causing damage to the tissue. Because the brain naturally moves with respect to the skull (e.g., due to pulsations from the heart), the bending angle of about 180° allows the flexible electrode array 208 to remain affixed (e.g., along X and Y directions) to the dural layer of the patient, enabling the first portion 602 (e.g., portion extended out of the hermetically sealed package 204) to pivot slightly with respect to rotation 1202, as illustrated in FIG. 10B.

FIG. 10A-2 illustrates another example process of forming the bent portion 902 featuring a 180-degree bend, in accordance with some embodiments. The flexible electrode array 208 is obtained (e.g., fabricated or assembled). Separately, an attachment portion 1020 that includes the hermetic package 204, the hermetic feedthrough 1008, the implant electronics 206, and the cover 218 (e.g., hermetic lid) is obtained (e.g., fabricated and/or assembled). The flexible electrode array 208 and the attachment portion 1020 are electrically connected with each other (e.g., by electrically connecting the bond pads 612 of the flexible electrode array 208 to the attachment portion 1020 via the hermetic feedthrough 1008). The back cover 1006 is applied, and the flexible electrode array 208 is bent about 180° about the bent portion 902 via a thermoforming process.

FIG. 10A-3 illustrates another example process of forming the bent portion 902 featuring a 180-degree bend, in accordance with some embodiments. The flexible electrode array 208 is obtained (e.g., fabricated or assembled) and a ~180-degree bend is formed by bending the flexible electrode array 208 about the bent portion 902 via a thermoforming process. Separately, attachment portion 1020 that includes the hermetic package 204, the hermetic feedthrough 1008, the implant electronics 206, and the cover 218 (e.g., hermetic lid) is fabricated and/or assembled. The flexible electrode array 208 is temporarily folded back (1022) at the bent portion 902. The attachment portion 1020 and the back cover 1006 are attached, and the flexible electrode array 208 is re-folded (e.g., re-bent) at the bent portion 902.

FIG. 10C illustrates another view of the implant device 110 with the flexible electrode array 208, in accordance with some embodiments. In some embodiments, a total thickness of the electrode array-including the electrodes, the flexible substrate, and the backing layer-has a range of about 0.2 mm to 1.0 mm, 0.2 mm to 0.9 mm, 0.2 mm to 0.8 mm, 0.2 mm to 0.7 mm, 0.2 mm to 0.6 mm, 0.3 mm to 1.0 mm, 0.3 mm to 0.9 mm, 0.3 mm to 0.8 mm, 0.3 mm to 0.7 mm, 0.3 mm to 0.6 mm, 0.4 mm to 1.0 mm, 0.4 mm to 0.9 mm, 0.4 mm to 0.8 mm, 0.4 mm to 0.7 mm, 0.4 mm to 0.6 mm, 0.5 mm to 1.0 mm, 0.5 mm to 0.9 mm, 0.5 mm to 0.8 mm, 0.5 mm to 0.7 mm, and 0.5 mm to 0.6 mm. In some embodiments, the total thickness of the electrode array has a lower limit of around 0.3 mm to ensure good contact with the brain surface of the patient. In some embodiments, the total thickness of the electrode array has an upper limit of 1.0 mm to avoid putting pressure on the brain.

In some embodiments, at least a portion of the flexible electrode array 208 is curved to match a brain curvature. For example, at least a portion of the array substrate can be adapted (e.g., by bending the flexible substrate) to conform to a curve. In some embodiments, a radius of curvature of the flexible electrode array 208 ranges from 60 mm to 80 mm, 60 mm to 90 mm, 60 mm to 100 mm, 60 mm to 110 mm, 60 mm to 120 mm, 70 mm to 80 mm, 70 mm to 90 mm, 70 mm to 100 mm, 70 mm to 110 mm, 70 mm to 120 mm, 80 mm to 90 mm, 80 mm to 100 mm, 80 mm to 110 mm, or 80 mm to 120 mm.

In some embodiments, a uniform radius of curvature is applied across the entire flexible electrode array 208. In some embodiments, the flexible electrode array 208 includes a first portion that is flat (i.e., with zero radius of curvature) and a second portion that is curved, where the second portion has a constant radius of curvature. In some embodiments, the flexible electrode array 208 includes two or more portions that are curved, and each curved portion has the same radius of curvature. In some embodiments, the flexible electrode array 208 includes two or more portions that are curved, and at least two of the two or more portions have different radii of curvature. In some embodiments, the flexible electrode array 208 includes at least two portions that are flat (i.e., with zero radius of curvature) and at least two portions that are curved, and each of the curved portions has a respective radius of curvature.

In some embodiments, the radius of curvature of the flexible electrode array 208 is selected so that it is “flat” enough such that the electrode array 208 can be inserted into the space between the dura and the brain fairly easily (e.g., if it is too curved, then the array could buckle instead of sliding into the gap between dura/brain) and yet “curved” enough such that it facilitates a slight downward pressure on the electrode array and improves electrode contact with the brain. In some embodiments, the curvature of the flexible electrode array is purposefully selected to be less than the curvature of the brain. When the curvature of the flexible electrode array perfectly matches the curvature of the brain, the electrode array may end up “floating” above the brain in situations where the backing layer is thin and does not generate sufficient downward force. In addition, slightly under-curving the electrode array relative to the curvature of the brain facilitates ease of insertion into the space between the dura and the brain.

As disclosed, in some embodiments, the ~180-degree bend at the bent portion 902 of the flexible electrode array 208 is used to couple the electrode array to an edge of the hermetically sealed package, route the bond-pad portion to the hermetic feedthrough 1008 while allowing at least part of the first portion 602 to extend beneath the dura. The bent portion 902 can provide a compact routing configuration that permits the second portion 604 to electrically couple to a hermetically sealed package 204 through a hermetic feedthrough. In some embodiments, the bend of about 180 degrees allows the flexible electrode array 208 to accommodate relative movement between the brain and the skull without causing undue stress on tissue or on the flexible electrode array 208. The bent portion 902 can further facilitate placement of the flexible electrode array 208 close to the dura through a slit in the dura and can permit the flexible electrode array 208 to be temporarily unfolded during insertion and subsequently re-folded after placement. In some embodiments, the bend geometry also supports desired placement, mechanical robustness, contact with the dura, and signal acquisition.

FIG. 11 illustrates another example process of forming the bent portion 902 featuring a 90-degree bend, in accordance with some embodiments. The flexible electrode array 208 shown in FIG. 11 is similar to that shown in FIG. 10, and differs primarily in that the electrodes 610 and the bond pads 612 are formed on different sides of the flexible electrode array 208. FIG. 11 shows that the electrodes 610 are formed on a first side 1102 and the bond pads 612 are formed on a second side 1104. The back cover 1006 is positioned on the first side 1002 of the flexible electrode array 208 and coupled to the bond pads 612 through the second portion 604 of the flexible electrode array 208. In some embodiments, the bent portion 902 of the flexible electrode array 208 is formed by bending the first portion 602 of the flexible electrode array 208 with respect to the second portion 604 of the flexible electrode array 208 at the bending axis 601 (e.g., Y axis) along a width W of the flexible electrode array 208. In some embodiments, the first portion 602 of the flexible electrode array 208 is contiguous to the second portion 604 of the flexible electrode array 208. In some embodiments, the bent portion is formed using a thermoforming process.

In some embodiments, the bent portion 902 of the flexible electrode array 208 is coupled to a side edge 1106 (e.g., a short edge) of the hermetically sealed package 204 that is perpendicular to a bottom edge (e.g., bottom edge 1010 in FIG. 10). In some embodiments, the bent portion 902 of the flexible electrode array 208 is configured to couple to the side edge 1106 of the hermetically sealed package 204 via the hermetic feedthrough 1008. In some embodiments, the bent portion 902 of the flexible electrode array 208 is configured to couple to the side edge 1106 of the hermetically sealed package 204 via the hermetic feedthrough 1008 at the second side 1104 of the bent portion 902 and configured to couple to the back cover 1006 at the first side 1102 of the bent portion 902. In some embodiments, the hermetic feedthrough 1008 is part of the hermetically sealed package 204 and configured to electrically connect the set of electronic components 316 to the flexible electrode array 208. In some embodiments, the back cover 1006 is part of the implant device 110.

In some embodiments, the bent portion 902 of the flexible electrode array 208 has a bending angle of about 90°. In some embodiments, the bent portion 902 of the flexible electrode array 208 has a bending angle of about 180°. In some embodiments, the bent portion 902 of the flexible electrode array 208 has a bending angle that ranges from about 90° to about 270°. Compared with the 180-degree flex, the 90-degree flex provides a different configuration in which the bent portion 902 of flexible electrode array 208 is oriented differently relative to the dura layer. In some embodiments, the choice of the bending angle can be determined by design specifications, such as placement of the flexible electrode array 208, mechanical robustness, thermal stability, contact with the underlying dural layer, and signal acquisition.

### Craniectomy Process

Various embodiments are directed to surgical processes of inserting a cranial implant device into an opening in a skull of a patient. Some embodiments are directed to a surgical process of inserting the cranial implant device that minimizes the opening of the dura mater layer of the patient. The dura mater layer (e.g., dural) protects brain and spinal cord. For example, the dura mater layer helps protect the brain and spinal cord from trauma. During an implant surgery, the dura mater (e.g., tough membrane covering the brain) is typically incised to expose the brain tissue for placement of an implant device, which may cause a defect to the dura mater layer and potentially lead to cerebrospinal fluid leakage or infection. Some embodiments are directed to methods that involve making a single slit in the dura mater layer and inserting (e.g., sliding) a flexible electrode array of the cranial implant device under the dura mater layer via the single slit. The single slit is minimal in size and can be strategically positioned in a way to reduce the risk of losing cerebral spinal fluid during the surgery.

FIG. 12A illustrates an example process 1200 of inserting the implant device 110 into an opening in a skull of a patient, in accordance with some embodiments. In some embodiments, the implant device 110 to be inserted includes the hermetically sealed package 204, the flexible electrode array 208 with the first portion 602, the second portion 604, and the bent portion, which are described above and not repeated for the sake of brevity. In some embodiments, the flexible electrode array 208 includes opening 616 to facilitate suturing, as described with reference to FIGS. 6A and 6B.

In FIG. 12A, a skull of the patient is opened and a slit 1204 (e.g., an incision) is made into a dura mater layer (e.g., dura layer) of a brain of the patient. A portion of the flexible electrode array 208 (e.g., about 50 mm to 90 mm), which includes at least a subset of the electrodes, is inserted under the dura mater layer of the patient via the slit 1204, such that the opening 616 of the flexible electrode array 208 is located at the slit 1204 (e.g., aligned with the slit). During the insertion process, the bending angle of the flexible electrode array 208 can be varied. After the portion of the flexible electrode array 208 has been inserted, the dura mater layer is sutured at the opening 616 (e.g., suturing position 1206-1) and/or at other positions such as suturing position 1206-2 and suturing position 1206-3. Subsequent to the suturing, the flexible electrode array 208 is further bent to about 180°, and the hermetically sealed package 204 of the implant device 110 covers the exposed dura mater layer. In some embodiments, the opening 616 is positioned immediately next to the slit 1204. FIG. 12B is a cross-sectional view depicting the incision on the dura and the insertion of the flexible electrode array 208, in accordance with some embodiments. FIG. 12C illustrates exemplary positions of the sutures (e.g., suturing positions) relative to the flexible electrode array according to some embodiments.

FIG. 12D illustrates contact of the implant device 110 with bone and bodily fluids of a human subject in accordance with some embodiments. In some embodiments, the frame 202 contacts with the bone (e.g., skull) of a human subject 102. In some embodiments, the flexible electrode array 208 contacts the brain tissue and cerebrospinal fluid (CSF) (e.g., including blood) of the human subject 102. In some embodiments, the hermetically sealed package 204 contacts the subcutaneous (e.g., scalp) of the human subject 102.

FIG. 13 is a flowchart for a method 1300 of inserting a cranial implant device (e.g., implant device 110) into an opening in a skull of a patient, in accordance with some embodiments. The cranial implant device 110 includes an electrode array (e.g., electrode array 208) having an opening (e.g., opening 616 in FIGS. 6A-6C) at an interior portion (e.g., first portion 602, or part of first and second portions 602 and 604 in FIGS. 6A-6C) of the electrode array 208.

The method 1300 includes making (1302) an incision (e.g., slit 1204 in FIG. 12A) into a dura mater layer (e.g., dura layer of a brain) of the patient.

The method 1300 includes inserting (1304) a first portion (e.g., portion 602), less than all, of the electrode array 208 under the dura mater layer of the patient via the incision, such that the opening 616 at the interior portion (e.g., first portion 602, or part of first and second portions 602 and 604) of the electrode array 208 is located at the dural incision.

In some embodiments, inserting the first portion of the electrode array under the dura mater layer of the patient via the incision includes aligning (1306) the opening with the incision

The method 1300 includes fastening (1308) the dura mater layer at the opening 616. In some embodiments, the opening 616 is positioned immediately next to the slit 1204. In some embodiments, the opening 616 is positioned proximate to the slit 1204. In some embodiments, fastening the dura mater layer includes applying (1310) one or more of suturing, clipping or other surgical methods of reapproximating the dura.

In some embodiments, the electrode array 208 includes a necking section (e.g., necking section 618 in FIG. 6B) having a width (e.g., W_{neck}) that is smaller than a width W of remaining section of the electrode array 208. The opening 616 is located in the necking section 618 of the electrode array 208. In some embodiments, the method 1300 includes fastening (1312) the dura mater layer at a portion of a perimeter (e.g., at suturing position 1206-2 and suturing position 1206-3) of the necking section 618. In some embodiments, the opening 616 is located between the first portion 602 and the necking section 618 of the second portion 604 of the electrode array 208.

In some embodiments, the opening includes (1314) a mesh material or a bioabsorbable material. Fastening the dura mater layer at the opening includes fastening the dura mater to the mesh material.

In some embodiments, the electrode array includes a bent portion. The method 1300 includes, prior to inserting the first portion of the electrode array, unbending (1316) the bent portion of the electrode array to form an unbent portion.

In some embodiments, the method 1300 includes, after fastening the dura mater layer at the opening: re-bending(1318) the unbent portion of the electrode array; and anchoring the cranial implant device to the skull of the patient via a plurality of screws.

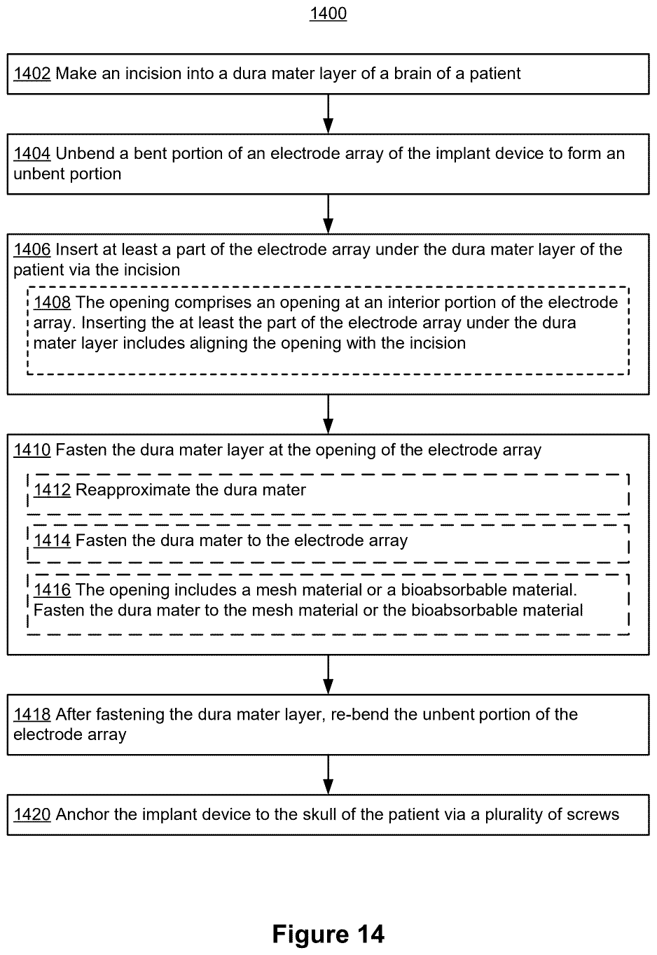

FIG. 14 a flowchart for a method 1400 of inserting a cranial implant device (e.g., implant device 110) into an opening in a skull of a patient, in accordance with some embodiments. The cranial implant device 110 includes an electrode array (e.g., electrode array 208) having a bent portion (e.g., bent portion 902 in FIGS. 9-12) and an opening (e.g., opening 616).

The method 1400 includes making (1402) an incision (e.g., slit 1204 in FIG. 12) into a dura mater layer (e.g., dura layer) of a brain of the patient.

The method 1400 includes unbending (1404) (e.g., straightening, applying a rotation 1202) the bent portion 902 of the electrode array 208 to form an unbent portion (e.g., bent portion 902 becoming substantially flat).

The method 1400 includes inserting (1406) at least a part of the electrode array 208 under the dura mater layer of the patient via the incision (e.g., slit 1204).

In some embodiments, the opening comprises (1408) an opening at an interior portion of the electrode array. For example, as illustrated in FIG. 6B, the opening 616 is located at an interior portion of the electrode array 208. Inserting the at least the part of the electrode array under the dura mater layer includes aligning the opening with the incision (e.g., placing the opening 616 immediately next to the incision or slit 1204).

The method 1400 includes fastening (1410) the dura mater layer at the opening of the electrode array. In some embodiments, fastening the dura mater layer includes reapproximating (1412) the dura mater. In some embodiments, fastening the dura mater layer includes fastening (1414) the dura mater to the electrode array. In some embodiments, the opening includes (1416) a mesh material or a bioabsorbable material. Fastening the dura mater layer includes fastening the dura mater to the mesh material or the bioabsorbable material.

The method 1400 includes after fastening the dura mater layer, re-bending (1418) the unbent portion of the electrode array 208.

The method 1400 includes anchoring (1420) the cranial implant device 110 to the skull of the patient via a plurality of screws (e.g., around edge(s) of the cranial implant device 110).

In some embodiments, the electrode array 208 includes a necking section (e.g., necking section 618) having a width W_{neck} that is smaller than a width of a remaining section (e.g., including the first portion 602 and the first section 604-1 of the second portion 604) of the electrode array 208. Inserting the at least a part of the electrode array 208 under the dura mater layer of the patient via the slit 1204 includes aligning the necking section with the slit 1204 (e.g., placing the opening 616 immediately next to the slit 1204.

Turning to some example embodiments:

In one aspect, a cranial implant device 110 (e.g., implant device 110) is configured to be inserted into an opening in a skull of a patient. The cranial implant device 110 includes an electrode array 208 (e.g., electrode array 208, 208A, 208B, 208C, 208D, or 208E) configured to be positioned on a brain surface of the patient. The electrode array 208 includes a flexible substrate 608 with electrode leads 614. The electrode array 208 further includes a plurality of electrodes 610 (e.g., electrode contacts) (e.g., electrode sensors 210) mounted on and protruding from the flexible substrate 608 such that at least some of the plurality of electrodes 610 are configured to be proximate to the brain surface of the patient when positioned in the patient. This is illustrated in, for example, FIGS. 2A, 2B, 6A-6C, 9, and 12.

In some embodiments, a plurality of sensors (e.g., electrode sensors 210 in FIG. 2A) are deposited on a surface of the flexible substrate 608.

In some embodiments, the electrode array 208 includes an additional layer 702 that is applied to the flexible substrate 608 and stands proud of the flexible substrate 608. The additional layer 702 includes a substrate portion 704 and a sensor layer (e.g., as electrodes 610). The plurality of electrodes 610 stand proud of the additional layer 702. This is illustrated in, for example, FIGS. 7A-7C.

In some embodiments, the flexible substrate 608 includes a biocompatible material. For example, in some embodiments, the flexible substrate 608 includes a liquid crystal polymer (LCP) material. For example, the LCP material can be fabricated as a thin film with mechanical robustness and thermal stability. In some embodiments, the LCP material of the flexible substrate 608 includes molecules with rigid and flexible monomeric units. The rigid monomeric units provide high temperature capability and good mechanical properties, while the flexible monomeric units provide the processability of the molecule chains.

In some embodiments, the plurality of electrodes comprises a biocompatible material. For example, in some embodiments, the plurality of electrodes are made of platinum. In some embodiments, the plurality of electrodes 610 are made of other material(s) such as tantalum, titanium, or gold.

In some embodiments, the electrode array 208 is configured to be positioned on a brain surface under a dura mater layer (e.g., subdural) of the patient. This is illustrated in, for example, FIGS. 2B and 12. In some embodiments, the electrode array 208 is configured to be positioned on a brain surface above a dura mater layer (e.g., epidural) of the patient.

In another aspect, a cranial implant device 110 (e.g., implant device 110) is configured to be inserted into an opening in a skull of a patient. The cranial implant device 110 includes an electrode array 208 (e.g., electrode array 208, 208A, 208B, 208C, 208D, or 208E) configured to be positioned on a brain surface of a patient. The electrode array 208 includes a plurality of electrodes on a first side 802 of the electrode array 208, configured to be positioned on the brain surface of the patient. The electrode array 208 includes a backing layer 622. The backing layer is adapted to reduce a space between a skull and a brain of the patient, thereby improving contact between the electrodes and the brain surface. In some embodiments, the backing layer is coupled to a second side 804 of the electrode array 208, opposite to the first side. This is illustrated in, for example, FIGS. 2B, 6C, 8A, 8B, and 12. In some embodiments, the electrode array 208 includes one or more intermediate layers coupled to the second side, and the backing layer may be coupled to the one or more intermediate layers, as shown and described in FIGS. 8C-1 to 8C-3.

In some embodiments, the backing layer 622 includes a silicone backing layer. In some embodiments, silicone increases the stiffness of the backing layer 622, such that the backing layer 622 reduces a gap between the dural layer and the brain of the patient and prevents movement of the flexible substrate 608 relative to the brain surface. This is illustrated in, for example, FIGS. 6C, 8A, and 8B.

In some embodiments, a thickness of the electrode array is around 0.25 millimeters (mm) to 1 mm. This is illustrated in, for example, FIGS. 8A and 8B. For example, in some embodiments, the electrode array includes a flexible substrate with electrodes and a backing layer, and a combined thickness of the flexible substrate with the electrodes and the backing layer is about 0.25 mm to 1 mm. In accordance with some embodiments, the combined thickness of the flexible substrate with the electrodes and the backing layer theoretically matches the size of the subdural space (e.g., the distance between the dura (stuck to the inside of the skull) and the brain surface).

In some embodiments, a thickness of the backing layer 622 is adjustable (e.g., variable) according to an anatomy of the patient.

In some embodiments, a thickness of the backing layer 622 is adjustable (e.g., variable) according to a respective region of the brain in which the cranial implant device 110 is configured to be positioned.

In some embodiments, the backing layer 622 includes an inflatable backing layer (e.g., inflatable backing layer 806). For example, the inflatable backing layer is adjustable for each patient, e.g., based on the specific spacing between the dura layer and the brain, to prevent movement of the electrode array 208.

In some embodiments, the backing layer 622 includes a compressible material.

In some embodiments, the backing layer 622 includes a cavity (e.g., internal space) that includes one or more materials, including one or more of: a flowable silicone elastomer, a plastic bead, a mineral oil, a saline solution, or a hydrogel.

In some embodiments, the electrode array 208 (e.g., flexible electrode substrate 608) includes one or more fins 624 (e.g., fin 624-1 and fin 624-2) (e.g., also referred to as flaps or wings) that extend from one or more respective edges (e.g., long edges) of the electrode array 208, toward the second side 804 (e.g., back side) of the electrode array 208. In some embodiments, the one or more fins 624 are included in the flexible substrate 608, e.g., the one or more fins 624 extend from the flexible substrate 608. This is illustrated in, for example, FIGS. 6C and 6D. In some embodiments, the one or more fins 624 are part of the backing layer 622.

In some embodiments, the one or more fins 624 are configured to be bent (e.g., folded) along the one or more edges (e.g., long edges) toward the second side 804 (e.g., back side) of the electrode array 208. In some embodiments, the bent one or more fins 624 are used to reduce the gap between the dura layer and the brain of the patient. This is illustrated in FIG. 6D.

In some embodiments, the one or more fins are configured to be bent using a thermoforming process.

In some embodiments, the electrode array 208 is configured to be positioned on the brain surface of a patient by inserting a portion of the electrode array 208 under the skull of the patient, proximate to a dura mater layer of the patient (e.g., by making the slit 1204 in the dura mater layer). The one or more fins 624 are configured to form a flat surface with the second side 804 (e.g., back side) of the electrode array 208 when the portion of the electrode array 208 is inserted (e.g., during a surgical insertion process). The one or more fins 624 are further configured to unfold toward the skull of the patient after the portion of the electrode array 208 has been inserted (e.g., thereby holding the electrode array 208 against the brain surface). This is illustrated in, for example, FIG. 6C.

In yet another aspect, a cranial implant device 110 (e.g., cranial implant device 110, implant device 110) includes a hermetically sealed package 204 (e.g., an “electronics can”) configured to be inserted into an opening in a skull of a patient. The cranial implant device 110 further includes a set of electronic components 206 enclosed within the hermetically sealed package 204. The cranial implant device 110 further includes a flexible electrode array 208 configured to be positioned on a brain surface of the patient. The flexible electrode array has a bent portion 902 that is configured to couple (e.g., attached) to an edge 904 (e.g., a side edge or a bottom edge) (of the hermetically sealed package 204, such that a subset of electrodes (e.g., the plurality of electrodes 610, or a subset of the plurality of electrodes 610) of the flexible electrode array 208 is positioned under the hermetically sealed package 204. This is illustrated in, for example, FIGS. 9-12.

In some embodiments, the bent portion 902 is formed by bending a first portion 602 of the flexible electrode array 208 with respect to a second portion 604 of the flexible electrode array 208 at a bending axis 601 along a width W of the flexible electrode array 208. The first portion 602 is contiguous to the second portion 604. In some embodiments, this bending technique allows the second portion 604 of the flexible electrode array 208 to be inserted into the hermetically sealed package 204 for electrical connections. In some embodiments, the bent portion 902 is the portion of the flexible electrode array 208 that includes bond pads (e.g., bond pads 612 in FIGS. 6A-6C). Stated another way, in some embodiments, the bent portion 902 includes the second portion 604 or at least a part of the second portion 604 of the flexible electrode array 208. This is illustrated in, for example, FIGS. 9-12.

In some embodiments, the bent portion 902 is formed via a thermoforming process.

In some embodiments, the bent portion 902 of the flexible electrode array 208 is coupled to a side edge 1106 (e.g., a short edge) of the hermetically sealed package 204 that is perpendicular to a bottom edge (e.g., bottom edge 1010 in FIG. 10). The bottom edge 1010 is facing the surface of the brain of the patient. This is illustrated in, for example, FIGS. 9 and 11.

In some embodiments, the bent portion 902 of the flexible electrode array 208 is coupled to a bottom edge 1010 (e.g., a long edge) of the hermetically sealed package 204. The bottom edge 1010 is facing the surface of the brain of the patient. This is illustrated in, for example, FIGS. 9 and 10A-1.

In some embodiments, the hermetically sealed package 204 includes a hermetic feedthrough 1008 for electrically connecting the set of electronic components 206 to the flexible electrode array 208 (e.g., to the plurality of bond pads 612 of the flexible electrode array 208). The bent portion 902 of the flexible electrode array 208 is configured to couple to the edge (e.g., bottom edge 1010 or side edge 1106) of the hermetically sealed package 204 via the hermetic feedthrough 1008. In some embodiments, the hermetic feedthrough 1008 is part of the hermetically sealed package 204 and configured to electrically connect the set of electronic components 316 to the flexible electrode array 208. This is illustrated in, for example, FIGS. 10 and 11.

In some embodiments, the cranial implant device 110 further includes a cover 1006 (e.g., back cover 1006). The bent portion 902 of the flexible electrode array 208 is configured to couple to the bottom edge 1010 of the hermetically sealed package 204 via the hermetic feedthrough 1008 at a first side 1002 of the bent portion 902 and configured to couple to the cover 1006 at a second side 1004 of the bent portion 902. Alternatively, in some embodiments, the bent portion 902 of the flexible electrode array 208 is configured to couple to the side edge 1106 of the hermetically sealed package 204 via the hermetic feedthrough 1008 at the second side 1004 of the bent portion 902 and configured to couple to the cover 1006 at the first side 1002 of the bent portion 902. This is illustrated in, for example, FIGS. 10A-1 and 11.

In some embodiments, the bent portion has a bending angle of about 90°. In some embodiments, a 90-degree flex (e.g., bending angle) provides a different configuration in which the bent portion 902 of flexible electrode array 208 is oriented differently relative to the dura layer. This is illustrated in, for example, FIG. 11.

In some embodiments, the bent portion has a bending angle of about 180°. In some embodiments, a 180-degree flex (e.g., bending angle) allows for movement of the flexible electrode array 208 without causing damage to the tissue. In some embodiments, the 180-degree flex allows the flexible electrode array 208 to be placed close to the dura layer through a slit (e.g., slit 1204 in FIG. 12). This is illustrated in, for example, FIG. 10A-1.

In some embodiments, the bent portion has a bending angle in a range of about 90° to 180°. In some embodiments, the bent portion has a bending angle in a range of about 90° to 270°. In some embodiments, the choice of the bending angle can be determined by design specifications, such as placement of the flexible electrode array 208, mechanical robustness, thermal stability, contact with the underlying dura layer, and signal acquisition.

In some embodiments, the second portion 604 of the flexible electrode array 208 includes an opening 616. The flexible electrode array 208 is configured to be inserted between a dural layer (e.g., dura mater) and a brain of the patient. The opening 616 is configured for suturing the flexible electrode array 208 onto the dura layer of the patient. In some embodiments, the opening 616 is at the second portion 604 and close to where the plurality of electrodes 610 are inserted. Alternatively, in some embodiments, the opening 616 is positioned between the first portion 602 and the second portion 604. Alternatively, in some embodiments, the opening 616 is positioned at the first portion 602. This is illustrated in, for example, FIGS. 6C, 9, and 12.

In some embodiments, the second portion 604 of the flexible electrode array 208 includes a first section 604-1 having a first width W₁ and a second section 604-2 (e.g., necking section 618) contiguous to the first section 604-1. The second section 604-2 has a second width W₂ that is smaller than the first width W₁. The opening 616 is positioned at the second section 604-2 of the second portion 604 of the flexible electrode array 208. This is illustrated in, for example, FIG. 6B.

In some embodiments, the flexible electrode array 208 includes a flexible substrate 608 and a plurality of electrodes 610 (e.g., arranged in an array) positioned on the flexible substrate 608. In some embodiments, the plurality of electrodes 610 are arranged in groups. Specifically, in some embodiments, the plurality of electrodes 610 includes one or more groups of electrodes (e.g., group 610-1 to group 610-m, where m is an integer greater than one). This is illustrated in, for example, FIG. 6A.

In some embodiments, the flexible substrate 608 includes a biocompatible polymer material such as polyimide (PI), polydimethylsiloxane (PDMS), and parylene C. In some embodiments, the flexible substrate 608 includes a liquid crystal polymer (LCP) material. For example, the LCP material can be fabricated as a thin film with mechanical robustness and thermal stability. In some embodiments, the LCP material of the flexible substrate 608 includes molecules with rigid and flexible monomeric units. The rigid monomeric units provide high temperature capability and good mechanical properties, while the flexible monomeric units provide the processability of the molecule chains.

In some embodiments, the flexible substrate 608 has a thickness of about 50 micrometers (μm) to 200 μm. For example, in some embodiments, the thickness of the flexible substrate is in the range of about 50 μm to 100 μm, 50 μm to 150 μm, 50 μm to 200 μm, 100 μm to 150 μm, 100 μm to 200 μm, or 100 μm to 200 μm.

In some embodiments, the flexible electrode array 208 further includes a silicone backing (e.g., backing layer 622 includes a silicone backing layer). The plurality of electrodes 610 are positioned on a first side 802 of the flexible substrate 608, and the silicone backing is positioned on a second side 804 of the flexible substrate 608, opposite to the first side 802. The first side 802 of the flexible substrate 608 (e.g., and the plurality of electrodes 610) are configured to physically contact a brain surface of the patient. In some embodiments, the first side 802 of the flexible substrate 608 (e.g., and the plurality of electrodes 610) are configured to proximate to a brain surface of the patient. This is illustrated in, for example, FIGS. 8A and 8B.

In some embodiments, the silicone backing (e.g., backing layer 622 includes a silicone backing layer) has a thickness of about 250 μm to 500 μm. This is illustrated in, for example, FIGS. 8A and 8B.

In some embodiments, the bent portion 902 of the flexible electrode array 208 is configured to be located above a dura layer (e.g., dura mater) of the skull of the patient. The second portion 604 of the flexible electrode array 208 is configured to be located below the dura layer of the skull of the patient. In some embodiments, the second portion 604 of the flexible electrode array 208 is inserted below the dura layer through a slit (e.g., slit 1204 in FIG. 12). This is illustrated in, for example, FIGS. 2A, 2B, and 12.

In some embodiments, a length (e.g., L) of the electrode array 208 is about 110 millimeters (mm) to 150 mm. For example, in some embodiments, the length L has a range of values of around 110 mm to 120 mm, 110 mm to 130 mm, 110 mm to 140 mm, 110 mm to 150 mm, 120 mm to 130 mm, 120 mm to 140 mm, 120 mm to 150 mm, 130 mm to 140 mm, or 130 mm to 150 mm.

In some embodiments, a width (e.g., W) of the electrode array 208 is about 30 mm to 40 mm. For example, in some embodiments, the width W has a range of values of around 30 mm to 32 mm, 30 mm to 35 mm, 30 mm to 38 mm, 30 mm to 40 mm, 32 mm to 36 mm, 32 mm to 38 mm, 32 mm to 40 mm, 34 mm to 36 mm, 34 mm to 38 mm, 34 mm to 40 mm, 36 to 38 mm, or 38 to 40 mm.

In some embodiments, the flexible electrode array 208 is configured to be positioned on the brain surface epidurally (e.g., outside the dura mater layer). In some embodiments, the flexible electrode array is configured to be positioned on the brain surface subdurally (inside the dura mater layer). This is illustrated in, for example, FIGS. 2A, 2B, and 12.

The methods disclosed herein comprise one or more steps or actions for achieving the described method. The method steps and/or actions may be interchanged with one another without departing from the scope of the claims. In other words, unless a specific order of steps or actions is required for proper operation of the method that is being described, the order and/or use of specific steps and/or actions may be modified without departing from the scope of the claims.

Turning to some example embodiments:
(A1) In accordance with some embodiments, a cranial implant device is configured to be inserted into an opening in a skull of a patient, the cranial implant device comprises an electrode array configured to be positioned on a brain surface of a patient, the electrode array including (i) a flexible substrate with electrode leads; and (ii) a plurality of electrodes mounted on and protruding from the flexible substrate such that at least some of the plurality of electrodes are configured to be proximate to the brain surface of the patient when positioned in the patient.
(A2) In some embodiments of A1, a plurality of sensors are deposited on a surface of the flexible substrate.
(A3) In some embodiments of any of A1-A2, (i) the electrode array includes an additional layer that is applied to the flexible substrate and stands proud of the flexible substrate, the additional layer including a substrate portion and a sensor layer; and (ii) the plurality of electrodes stand proud of the additional layer.
(A4) In some embodiments of any of A1-A3, the flexible substrate comprises a biocompatible material.
(A5) In some embodiments of any of A1-A4, the flexible substrate comprises a liquid crystal polymer (LCP) material.
(A6) In some embodiments of any of A1-A5, the plurality of electrodes comprises a biocompatible material.
(A7) In some embodiments of any of A1-A6, the plurality of electrodes are made of platinum.
(A8) In some embodiments of any of A1-A7, the electrode array is configured to be positioned on a brain surface above a dura mater layer.
(A9) In some embodiments of any of A1-A8, the electrode array is configured to be positioned on a brain surface under a dura mater layer.
(B1) In accordance with some embodiments, a cranial implant device is configured to be inserted into an opening in a skull of a patient. The cranial implant device comprises: (1) an electrode array configured to be positioned in a sub-dural space, on a brain surface of a patient, the electrode array including: (1a) a plurality of electrodes on a first side of the electrode array configured to be positioned on the brain surface of the patient; and (1b) a backing layer, wherein the backing layer is adapted to reduce a space between a skull and the brain of the patient thereby improving contact between the electrodes and the brain surface. In some embodiments, the backing layer is coupled to a second side of the electrode array, opposite to the first side. In some embodiments, and as illustrated and described in FIGS. 8C-1 to 8C-3, one or more intermediate layers, different from the backing layer, may be coupled to the flexible substrate, and the backing layer is coupled to at least one of the one or more intermediate layers.
(B2) In some embodiments of B1, the backing layer comprises a silicone backing layer.
(B3) In some embodiments of any of B1-B2, wherein a thickness of the electrode array is around 0.25 millimeters (mm) to 1 mm. For example, in some embodiments, the electrode array includes a flexible substrate with electrodes and a backing layer, and a combined thickness of the flexible substrate with the electrodes and the backing layer is about 0.25 mm to 1 mm.
(B4) In some embodiments of any of B1-B3, a thickness of the backing layer is adjustable according to an anatomy of the patient.
(B5) In some embodiments of any of B1-B4, a thickness of the backing layer is adjustable according to a respective region of the brain in which the cranial implant device is configured to be positioned.
(B6) In some embodiments of any of B1-B5, the backing layer comprises an inflatable backing layer.
(B7) In some embodiments of any of B1-B5, the backing layer comprises a compressible material.
(B8) In some embodiments of any of B1-B5, the backing layer includes a cavity that includes one or more materials, including one or more of: a flowable silicone elastomer, a plastic bead, a mineral oil, a saline solution, or a hydrogel.
(B9) In some embodiments of any of B1-B8, wherein the electrode array comprises one or more fins that extend from one or more respective edges of the electrode array, toward a second side of the electrode array, opposite to the first side of the electrode array.
(B10) In some embodiments of B9, wherein the one or more fins are configured to be bent along the one or more edges toward the second side of the electrode array.
(B11) In some embodiments of any of B9-B10, wherein the one or more fins are configured to be bent using a thermoforming process.
(B12) In some embodiments of any of B9-B11, wherein: (1) the electrode array is configured to be positioned on the brain surface of a patient by inserting a portion of the electrode array under the skull of the patient, proximate to a dura mater layer of the patient; and (2) the one or more fins are configured to: (2a) lie flat against the second side of the electrode array when the portion of the electrode array is inserted; and (2b) unfold toward the skull of the patient after the portion of the electrode array has been inserted.
(B13) In some embodiments of any of B1-B12, wherein at least a portion of the electrode array is curved.
(B14) In some embodiments of B13, wherein a radius of curvature is between 60 mm and 120 mm.
(B15) In some embodiments of B13, wherein a radius of curvature is between 80 mm and 120 mm.
(C1) In accordance with some embodiments, a cranial implant device comprises: (1) a hermetically sealed package configured to be inserted into an opening in a skull of a patient; (2) a set of electronic components enclosed within the hermetically sealed package; and (3) a flexible electrode array configured to be positioned on a brain surface of the patient, the flexible electrode array including (3a) a flexible substrate with electrode leads; (3b) a first portion positioned under and electrically coupled to a bottom surface of the hermetically sealed package; (3c) a second portion having a plurality of electrodes mounted on the surface of the flexible substrate; and (3d) a bent portion configured to bend the first portion with respect to the second portion such that a subset of electrodes of the flexible electrode array is positioned under a back cover of the implant device.
(C2) In some embodiments of C1, the bent portion bends the first portion of the flexible electrode array with respect to the second portion of the flexible electrode array at a bending axis along a width of the flexible electrode array.
(C3) In some embodiments of any of C1-C2, the bent portion is formed via a thermoforming process.
(C4) In some embodiments of any of C1-C3, the cranial implant device further comprises a layer of silicone material placed on a surface of the flexible electrode array where the flexible electrode array meets a frame of the implant device.
(C5) In some embodiments of any of C1-C4, wherein the bent portion of the flexible electrode array is coupled to a bottom edge of the hermetically sealed package that is facing the brain surface.
(C6) In some embodiments of any of C1-C5, wherein the hermetically sealed package includes a hermetic feedthrough for electrically connecting the set of electronic components to the flexible electrode array; and the bent portion of the flexible electrode array is configured to couple to the edge of the hermetically sealed package via the hermetic feedthrough.
(C7) In some embodiments of any of C1-C6, wherein the bent portion has a bending angle of about 180°.
(C8) In some embodiments of any of C1-C6, wherein the bent portion has a bending angle in a range of about 90° to 180°.
(C9) In some embodiments of any of C1-C8, wherein: (i) the second portion of the flexible electrode array includes an opening; (ii) the flexible electrode array is configured to be inserted between a dura layer and a brain of the patient; and (iii) the opening is configured for suturing the flexible electrode array onto the dura layer of the patient.
(C10) In some embodiments of C9, wherein (i) the second portion of the flexible electrode array includes a first section having a first width and a second section contiguous to the first section, the second section having a second width that is smaller than the first width; and (ii) the opening is positioned at the second section.
(C11) In some embodiments of any of C1-C10, wherein the flexible electrode array includes a flexible substrate and a plurality of electrodes positioned on the flexible substrate.
(C12) In some embodiments of any of C1-C11, wherein the flexible substrate comprises a biocompatible polymer material.
(C13) In some embodiments of any of C1-C12, wherein the flexible substrate comprises a liquid crystal polymer (LCP) material.
(C14) In some embodiments of any of C1-C13, wherein the flexible substrate has a thickness of about 100 micrometers (μm) to 200 μm.
(C15) In some embodiments of any of C1-C14, wherein: (i) the flexible electrode array further includes a silicone backing; (ii) the plurality of electrodes are positioned on a first side of the flexible substrate; and (iii) the silicone backing is positioned on a second side of the flexible substrate, opposite to the first side, wherein the first side of the flexible substrate are configured to physically contact a brain surface of the patient.
(C16) In some embodiments of any of C1-C15, wherein the silicone backing has a thickness of about 250 μm to 500 μm.
(C17) In some embodiments of any of C1-C16, wherein: (i) the bent portion of the flexible electrode array is configured to be located above a dura layer of the skull; and (ii) the second portion of the flexible electrode array is configured to be located below the dura layer of the skull.
(C18) In some embodiments of any of C1-C17, wherein a length of the electrode array is about 20 millimeters (mm) to 120 mm.
(C19) In some embodiments of any of C1-C18, wherein a width of the electrode array is about 28 mm to 32 mm.
(C20) In some embodiments of any of C1-C19, wherein the flexible electrode array is configured to be positioned on the brain surface epidurally.
(C21) In some embodiments of any of C1-C20, wherein the flexible electrode array is configured to be positioned on the brain surface subdurally.
(D1) In accordance with some embodiments, a method of inserting a cranial implant device into an opening in a skull of a patient, where the cranial implant device includes an electrode array having an opening at an interior portion of the electrode array, comprises: (1) making an incision into a dura mater later of the patient; (2) inserting a portion, less than all, of the electrode array under the dura mater layer of the patient via the incision, such that the opening at the interior portion of the electrode array is located at the dural incision; and (3) fastening the dura mater later at the opening.
(D2) In some embodiments of D1, wherein fastening the dura mater includes applying one or more of suturing, clipping or other surgical methods of reapproximating the dura.
(D3) In some embodiments of any of D1-D2, wherein the electrode array includes a necking section having a width that is smaller than a width of a remaining section of the electrode array; the opening is located in the necking section of the electrode array; and the method further includes fastening the dura mater layer at a portion of a perimeter of the necking section.
(D4) In some embodiments of any of D1-D3, the electrode array includes a bent portion; and the method further comprises: (1) prior to inserting the first portion of the electrode array, unbending the bent portion of the electrode array to form an unbent portion; and (2) after fastening the dura mater layer at the opening: (2a) re-bending the unbent portion of the electrode array; and (2b) anchoring the cranial implant device to the skull of the patient via a plurality of screws.
(D5) In some embodiments of any of D1-D4, wherein inserting the first portion of the electrode array under the dura mater layer of the patient via the incision includes aligning the opening with the incision.
(D6) In some embodiments of any of D1-D5, wherein the opening includes a mesh material or a bioabsorbable material; and fastening the dura mater layer at the opening includes fastening the dura mater to the mesh material.
(E1) In accordance with some embodiments, a method of inserting a cranial implant device into an opening in a skull of a patient, the cranial implant device including an electrode array having a bent portion, comprises: (i) making a slit into a dura mater layer of a brain of the patient; (ii) unbending the bent portion of the electrode array to form an unbent portion; (iii) inserting at least a part of the electrode array under the dura mater layer of the patient via the slit; (iv) fastening the dura mater layer; (v) re-bending the unbent portion of the electrode array; and (vi) anchoring the cranial implant device to the skull of the patient via a plurality of screws.
(F1) In accordance with some embodiments, a method of inserting a cranial implant device into an opening in a skull of a patient, the cranial implant device including an electrode array having a bent portion and an opening, comprises: (1) prior to inserting at least a part of the electrode array into a dura mater layer of the patient via an incision, unbending the bent portion of the electrode array to form an unbent portion; (2) inserting the at least the part of the electrode array into the dura mater layer (3) fastening the dura mater layer at the opening; (4) re-bending the unbent portion of the electrode array, and (5) anchoring the cranial implant device to the skull of the patient via a plurality of screws.
(F2) In some embodiments of F1, wherein the opening of the electrode array comprises an opening at an interior portion of the electrode array; and inserting the at least the part of the electrode array under the dura mater layer of the patient via the incision includes aligning the opening with the incision.
(F3) In some embodiments of any of F1-F2, wherein fastening the dura mater includes reapproximating the dura mater.
(F4) In some embodiments of any of F1-F3, wherein fastening the dura mater includes fastening the dura mater to the electrode array.
(F5) In some embodiments of any of F1-F4, the electrode array includes an opening at an interior portion of the electrode array; the opening includes a mesh material or a bioabsorbable material; and fastening the dura mater includes fastening the dura mater to the mesh material or the bioabsorbable material.

It will be understood that, although the terms “first,” “second,” etc. may be used herein to describe various elements, these elements should not be limited by these terms. These terms are only used to distinguish one element from another. The terminology used herein is for the purpose of describing particular embodiments only and is not intended to be limiting of the claims. As used in the description of the embodiments and the appended claims, the singular forms “a,” “an” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

As used herein, the term “plurality” denotes two or more. For example, a plurality of components indicates two or more components. The term “determining” encompasses a wide variety of actions and, therefore, “determining” can include calculating, computing, processing, deriving, investigating, looking up (e.g., looking up in a table, a database or another data structure), ascertaining and the like. Also, “determining” can include receiving (e.g., receiving information), accessing (e.g., accessing data in a memory) and the like. Also, “determining” can include resolving, selecting, choosing, establishing and the like.

The phrase “based on” does not mean “based only on,” unless expressly specified otherwise. In other words, the phrase “based on” describes both “based only on” and “based at least on.”

As used herein, the term “exemplary” means “serving as an example, instance, or illustration,” and does not necessarily indicate any preference or superiority of the example over any other configurations or embodiments.

As used herein, the term “and/or” encompasses any combination of listed elements. For example, “A, B, and/or C” entails each of the following possibilities: A only, B only, C only, A and B without C, A and C without B, B and C without A, and a combination of A, B, and C.

The terminology used in the description of the invention herein is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The foregoing description, for the purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the invention and its practical applications, to thereby enable others skilled in the art to best utilize the invention and various embodiments with various modifications as are suited to the particular use contemplated.

What is claimed is:

1. A cranial implant device, comprising:
a hermetically sealed package configured to be inserted into an opening in a skull of a patient;
a set of electronic components enclosed within the hermetically sealed package; and
a flexible electrode array configured to be positioned on a brain surface of the patient, the flexible electrode array including:
a flexible substrate with electrode leads;
a first portion positioned under and electrically coupled to a bottom surface of the hermetically sealed package;
a second portion having a plurality of electrodes mounted on a surface of the flexible substrate; and
a bent portion configured to bend the first portion with respect to the second portion such that a subset of electrodes of the flexible electrode array is positioned under a back cover of the cranial implant device.

2. The cranial implant device of claim 1, wherein the bent portion bends the first portion of the flexible electrode array with respect to the second portion of the flexible electrode array at a bending axis along a width of the flexible electrode array.

3. The cranial implant device of claim 1, wherein the bent portion is formed via a thermoforming process.

4. The cranial implant device of claim 1, further comprising:
a layer of silicone material placed on a surface of the flexible electrode array where the flexible electrode array meets a frame of the cranial implant device.

5. The cranial implant device of claim 1, wherein the bent portion has a bending angle of about 180°.

6. The cranial implant device of claim 1, wherein the bent portion has a bending angle in a range of about 90° to 180°.

7. The cranial implant device of claim 1, wherein:
the second portion of the flexible electrode array includes an opening;
the flexible electrode array is configured to be inserted between a dura layer and a brain of the patient; and
the opening is configured for suturing the flexible electrode array onto the dura layer of the patient.

8. The cranial implant device of claim 1, wherein:
the second portion of the flexible electrode array includes a first section having a first width and a second section contiguous to the first section, the second section having a second width that is smaller than the first width; and
the opening is positioned at the second section.

9. The cranial implant device of claim 1, wherein the flexible substrate comprises a biocompatible polymer material.

10. The cranial implant device of claim 1, wherein the flexible substrate comprises a liquid crystal polymer (LCP) material.

11. The cranial implant device of claim 1, wherein the flexible substrate has a thickness of about 100 micrometers (μm) to 200 μm.

12. The cranial implant device of claim 1, wherein:
the bent portion of the flexible electrode array is configured to be located above a dura layer of the skull; and
the second portion of the flexible electrode array is configured to be located below the dura layer of the skull.

13. The cranial implant device of claim 1, wherein a length of the flexible electrode array is about 20 millimeters (mm) to 120 mm.

14. The cranial implant device of claim 1, wherein a width of the flexible electrode array is about 28 mm to 32 mm.

15. The cranial implant device of claim 1, wherein the flexible electrode array is configured to be positioned on the brain surface subdurally.

16. A method of inserting a cranial implant device into an opening in a skull of a patient, the cranial implant device including an electrode array having an opening at an interior portion of the electrode array, the method comprising:
making an incision into a dura mater layer of the patient;
inserting a first portion, less than all, of the electrode array under the dura mater layer of the patient via the incision, such that the opening at the interior portion of the electrode array is located at the dural incision; and
fastening the dura mater layer at the opening.

17. The method of claim 16, wherein:
the electrode array includes a necking section having a width that is smaller than a width of a remaining section of the electrode array;
the opening is located in the necking section of the electrode array; and
the method further includes fastening the dura mater layer at a portion of a perimeter of the necking section.

18. The method of claim 16, wherein:
the electrode array includes a bent portion; and
the method further comprises:
prior to inserting the first portion of the electrode array, unbending the bent portion of the electrode array to form an unbent portion; and
after fastening the dura mater layer at the opening:
re-bending the unbent portion of the electrode array; and
anchoring the cranial implant device to the skull of the patient via a plurality of screws.

19. The method of claim 16, wherein:
inserting the first portion of the electrode array under the dura mater layer of the patient via the incision includes aligning the opening with the incision.

20. The method of claim 16, wherein:
the opening includes a mesh material or a bioabsorbable material; and
fastening the dura mater layer at the opening includes fastening the dura mater to the mesh material.
